SEQUENCE LISTING (29 sequences)

SEQ ID NO: 1 - RNA, 20 nt, synthetic construct
  source 1..20: mol_type = other RNA
gagtagcgcgagcacagcta

SEQ ID NO: 2 - RNA, 20 nt, synthetic construct
  source 1..20: mol_type = other RNA
agggtaggagagactcacgc

SEQ ID NO: 3 - RNA, 20 nt, synthetic construct
  source 1..20: mol_type = other RNA
ggccgagatgtctcgctccg

SEQ ID NO: 4 - RNA, 20 nt, synthetic construct
  source 1..20: mol_type = other RNA
gagcgtgtgactagctgtac

SEQ ID NO: 5 - DNA, 160 bp, synthetic construct
  source 1..160: mol_type = other DNA
attcctgaagctgacagcattcgggccgagatgtccaggtcctaatgattagctgtgctcgccctgctctctctgtctggcctggaggctattcagcgtgagtctctcctaccctcccgctctggtccttcctctcccgctctgcaccctctgtggccct

SEQ ID NO: 6 - DNA, 340 bp, synthetic construct
  source 1..340: mol_type = other DNA
aaaacgggaaagtccctctctctaacctggcactgcgtcgctggcttggagacaggtgacggtccctgcgggccttgtcctgattggctgggcacgcgtttaatataagtggaggcgtcgcgctggcgggcattcctgaagctgacagcattcgggccgagatgtcgtagagcgtgtgactagctgtactggagctgtgaagctaatccggtctggaagccattcagcgtgagtctctcctaccctcccgctctggtccttcctctcccgctctgcaccctctgtggccctcgctgtgctctctcgctccgtgacttcccttctccaagttctccttggt

SEQ ID NO: 7 - RNA, 20 nt, synthetic construct
  source 1..20: mol_type = other RNA
aagtcaacttcaatgtcgga

SEQ ID NO: 8 - RNA, 20 nt, synthetic construct
  source 1..20: mol_type = other RNA
agtcacatggttcacacggc

SEQ ID NO: 9 - RNA, 20 nt, synthetic construct
  source 1..20: mol_type = other RNA
acttgtctttcagcaaggac

SEQ ID NO: 10 - RNA, 20 nt, synthetic construct
  source 1..20: mol_type = other RNA
cctagatccaatagtagagt

SEQ ID NO: 11 - RNA, 20 nt, synthetic construct
  source 1..20: mol_type = other RNA
ggtcacgtggttcaccctac

SEQ ID NO: 12 - DNA, 281 bp, synthetic construct
  source 1..281: mol_type = other DNA
aaatgtaaacacttggtgcctgatatagcttgacaccaagttagccccaagtgaaataccctggcaatattaatgtgtcttttcccgatattcctcaggtaccccctagatccaatagtagagtaggtgaccagcctagaacggagcctgtagggtgaaccacgtgaccctgtaacagtggggtaagtcttacattcttttgtaagctgctgaaagttgtgtatgagtagtcatatcataaagctgctttgatataaaaaaggtctatggccatactaccc

SEQ ID NO: 13 - RNA, 20 nt, synthetic construct
  source 1..20: mol_type = other RNA
atggagttggggcccctaga

SEQ ID NO: 14 - RNA, 20 nt, synthetic construct
  source 1..20: mol_type = other RNA
ctaccacttctatgaccaga

SEQ ID NO: 15 - RNA, 20 nt, synthetic construct
  source 1..20: mol_type = other RNA
gtggcacactgtgagctgcc

SEQ ID NO: 16 - RNA, 20 nt, synthetic construct
  source 1..20: mol_type = other RNA
gtgacccctataatgagacc

SEQ ID NO: 17 - RNA, 20 nt, synthetic construct
  source 1..20: mol_type = other RNA
cagttcccagtgtgctacca

SEQ ID NO: 18 - DNA, 149 bp, synthetic construct
  source 1..149: mol_type = other DNA
ttctgcctctttccaacaccctgtgaggtgactgagcattgtcttccctcccaggcagttcccagtgtgctaccatggagttgtgacccctataatgagacctggctggagaagaagagattgagctctactcaggtgggccctcctcc

SEQ ID NO: 19 - RNA, 20 nt, synthetic construct
  source 1..20: mol_type = other RNA
aggctgttgtgtgacatgga

SEQ ID NO: 20 - DNA, 20 bp, synthetic construct
  source 1..20: mol_type = other DNA
tgactgatgtaagactagta

SEQ ID NO: 21 - DNA, 198 bp, synthetic construct
  source 1..198: mol_type = other DNA
aaatttccttcttcatccaagggacttttcctcccagaacccgacacagacaccatcaactgcgaccagttcagctgactgatgtaagactagtaaggtgatgaagagaccagggaggcttatgccaatatcggtgaggaagcacctgagcccagaaaaggacaatcaagggcaagagttctttgctgccacttgtca

SEQ ID NO: 22 - DNA, 160 bp, synthetic construct
  source 1..160: mol_type = other DNA
attcctgaagctgacagcattcgggccgagatgtctcgctccgtggccttagctgtgctcgcgctactctctctttctggcctggaggctatccagcgtgagtctctcctaccctcccgctctggtccttcctctcccgctctgcaccctctgtggccct

SEQ ID NO: 23 - DNA, 476 bp, synthetic construct
  source 1..476: mol_type = other DNA
aaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccc

SEQ ID NO: 24 - DNA, 215 bp, synthetic construct
  source 1..215: mol_type = other DNA
ttctgcctctttccaacaccctgtgaggtgactgagcattgtcttccctcccaggcagctcacagtgtgccaccatggagttggggcccctagaaggtggctacctggagcttcttaacagcgatgctgaccccctgtgcctctaccacttctatgaccagatggacctggctggagaagaagagattgagctctactcaggtgggccctcctcc

SEQ ID NO: 25 - DNA, 198 bp, synthetic construct
  source 1..198: mol_type = other DNA
aaatttccttcttcatccaagggacttttcctcccagaacccgacacagacaccatcaactgcgaccagttcagcaggctgttgtgtgacatggaaggtgatgaagagaccagggaggcttatgccaatatcggtgaggaagcacctgagcccagaaaaggacaatcaagggcaagagttctttgctgccacttgtca

SEQ ID NO: 26 - DNA, 57 bp, synthetic construct
  source 1..57: mol_type = other DNA
gtcgtggtttgtctggtcaaccaccgcggtctcagtggtgtacggtacaaaccccga

SEQ ID NO: 27 - DNA, 50 bp, synthetic construct
  source 1..50: mol_type = other DNA
tcggccggcttgtcgacgacggcggtctccgtcgtcaggatcatccgggc

SEQ ID NO: 28 - DNA, 42 bp, synthetic construct
  source 1..42: mol_type = other DNA
gtgccccaactggggtaacctttgagttctctcagttggggg

SEQ ID NO: 29 - DNA, 45 bp, synthetic construct
  source 1..45: mol_type = other DNA
tgcgggtgccagggcgtgcccttgggctccccgggcgcgtactcc

(19) United States
(12) Patent Application Publication
(10) Pub. No.: US 2026/0055370 A1
(43) Pub. Date: Feb. 26, 2026

(54) RE-EDITABLE TEMPLATES, CELLS, COMPOSITIONS AND METHODS OF MAKING

(71) Applicant: TAKEDA PHARMACEUTICAL COMPANY LIMITED, Osaka-shi (JP)

(72) Inventors: John STEELE, Cambridge, MA (US); Luis BORLADO, Cambridge, MA (US)

(21) Appl. No.: 19/377,673

(22) Filed: Nov. 3, 2025

Related U.S. Application Data
(63) Continuation of application No. PCT/IB2024/054536, filed on May 9, 2024.
(60) Provisional application No. 63/465,352, filed on May 10, 2023.

(51) Int. Cl. C12N 5/0783; C07K 14/705; C12N 5/074; C12N 5/0781; C12N 5/0786; C12N 5/10; C12N 9/22; C12N 9/78; C12N 15/113; C12N 15/90

(52) U.S. Cl. CPC C12N 5/0636; C07K 14/705; C12N 5/0635; C12N 5/0645; C12N 5/0646; C12N 5/0696; C12N 5/10; C12N 9/226; C12N 9/78; C12N 15/1138; C12N 15/907; C12Y 305/04001; C12Y 305/04002; C07K 2319/81; C12N 2310/20; C12N 2510/00

(57) ABSTRACT

The present invention provides, among other things, methods of engineering a re-editable target locus in a cell, comprising inserting an exogenous re-editable template sequence to a target locus and methods of engineering a re-editable cell comprising a re-editable template sequence flanked by two homology arms such that the re-editable template sequence is inserted into a target locus by homologous recombination, and methods of using the same. In some aspects, provided is a re-editable template sequence, wherein the template sequence has no substantial sequence similarity to any region in the genome, and wherein the template sequence is recognizable by a genome editing system.

### CROSS-REFERENCE TO RELATED APPLICATIONS

This application is a Continuation of International Application No. PCT/IB2024/054536, filed on May 9, 2024, which claims priority to, and the benefit of, U.S. Provisional Patent Application Ser. No. 63/465,352 filed on May 10, 2023; the contents of each of which are incorporated herein by reference in their entireties.

### INCORPORATION BY REFERENCE OF SEQUENCE LISTING

The instant application contains a sequence listing which has been submitted electronically in XML format and is hereby incorporated by reference in its entirety. The sequence listing file entitled MIL-024WO1.XML, was created on Apr. 23, 2024, which is 18,368,504 bytes in size.

### BACKGROUND

Genome editing technologies based on programmable nucleases such as meganucleases, zinc finger nucleases (ZFN), transcription activator-like effector nucleases (TALEN), and clustered regularly interspaced short palindromic repeat (CRISPR)-associated RNA-guided nucleases (e.g. Cas9, Cas12a, etc.) are powerful tools to achieve therapeutic genome editing at precise loci in diseased cells and tissues, resulting in the removal or correction of deleterious mutations or the insertion of protective mutations.

While ZFN, TALEN, and meganucleases achieve specific DNA binding via protein-DNA interactions, Cas and related nucleases are targeted to specific sites by an RNA guide molecule that base-pairs directly with the target locus. However, despite the benefits of precise genome targeting, these genome editing technologies have limitations such as unwanted cleavage at previously edited sites as well as off-target sites that remain of concern.

Sequence-specific recombinases and integrases, e.g., serine recombinases with specificity for unique attachment sites (attP and attB) are other useful tools to expand genome engineering strategies as they recognize and target specific genomic sequences thereby repairing genetic mutations or carrying out site-specific integration of functional genes. However, insertions of DNA sequences in the genome are not scarless and residual sequences from the integration event remain.

Programmable epigenome editing, on the other hand, does not require DNA breaks thereby eliminating cellular toxicity associated with gene editing, and it is also tunable and reversible. However, programmable epigenome editing requires constitutive expression of dCas-fusion proteins, making it challenging for therapeutic cell and organismal editing.

### SUMMARY OF THE INVENTION

The present invention provides a flexible genome editing technology that is precise and specific for target loci, but also allows for re-engineering and further manipulation of edited loci to achieve different phenotypes or re-engineer cells of specific phenotypes within a short time for therapeutic use. As described in greater detail below, and in the Examples, the present invention provides a ‘Re-edit Technology’ comprising methods of engineering re-editable target loci and cells with a re-editable template. Provided herein are methods of designing a re-editable template that is unique and recognizable by specific genome editing systems.

The present invention provides, among other things, methods of engineering a re-editable target locus in a cell, comprising inserting an exogenous re-editable template sequence to a target locus, wherein the template sequence has no substantial sequence similarity to any region in the genome, and wherein the template sequence is recognizable by a genome editing system. The present invention also provides, among other things, methods of engineering a re-editable cell comprising a unique re-editable template sequence flanked by two homology arms such that the re-editable template sequence is inserted into the target locus by homologous recombination, and methods of using the same.

In some aspects, provided herein is a re-editable template sequence, wherein the template sequence has no substantial sequence similarity to any region in the genome, and wherein the template sequence is recognizable by a genome editing system. The re-editable template of the present invention allows subsequent targeted modification of cells by a different genome editing nuclease than the one used to insert the re-editable template. The re-editable template and methods of the present invention facilitate rapid development of multiple different types of mutations in platform cells in a plug-and-play approach for treating multiple different indications.

Since the re-editable template of the present invention is designed to have no substantial sequence similarity to any region in the genome (for example, wherein another coding region in the genome recognizable by a guide RNA or another nuclease system contains at least 3 mismatches), off-target editing is minimized or eliminated, and the methods provided herein thus modify a locus to be uniquely editable using one or more genome editing tools, e.g. CRISPR-Cas, transcription activator-like effector nucleases (TALEN), or Zinc-finger nucleases (ZFN). In some embodiments, in a CRISPR-Cas system, gRNAs with established on- and off-target profiles are used to modify target loci accurately. In some embodiments, the re-editable locus is editable by another gRNA but not the same gRNA as used to insert the re-editable template. In some embodiments, a wild-type gene is knocked out by insertion of the re-editable template.

As described in the Examples section, in some embodiments, HLA Class I and II genes are knocked out and exemplary modified target loci, e.g. B2M and CIITA are integrated with specific re-editable templates used to induce a double-stranded break by a nuclease and thus able to be manipulated subsequently. In some embodiments, in the subsequent manipulation, a protein coding gene is integrated in the template sequence, for example, to protect cells from recognition by NK cells (cloaking gene), to terminate cells in the presence of an inducing molecule (safety switch), or express one or more proteins that may increase the efficacy of the product (e.g. CAR cassette). In some embodiments, a platform iPSC line is generated which is easily modifiable due to integration of a re-editable template with the advantage of reducing manufacturing time in the GMP suite for providing a universal off-the-shelf therapeutic iPSC or iPSC-derived cells for treating multiple disorders, thereby reducing costs. Briefly, the present invention is summarized as follows:

In some aspects, provided herein is a method of engineering a re-editable target locus in a cell, comprising inserting an exogenous re-editable template sequence to a target locus, wherein the template sequence has no substantial sequence similarity to any region in the genome, and wherein the template sequence is recognizable by a genome editing system.

In some aspects, provided herein is a method of engineering a re-editable cell, comprising introducing an exogenous re-editable template sequence flanked by two homology arms such that the re-editable template sequence is inserted into the target locus by homologous recombination, wherein the template sequence has no substantial sequence similarity to any region in the genome, and wherein the template sequence is recognizable by a genome editing system.

In some embodiments, the genome editing system is a CRISPR-Cas-related Nuclease system, a Transcription Activator-Like Effector Nuclease (TALEN) system, or a Zinc-Finger Nuclease (ZFN) system. In some embodiments, the genome editing system is a CRISPR-Cas-related Nuclease system. In some embodiments, the genome editing system is a Transcription Activator-Like Effector Nuclease (TALEN) system. In some embodiments, the genome editing system is a Zinc Finger Nuclease system (ZFN) system.

In some embodiments, the Cas protein is Cas9, Cas12a or Cas12b. In some embodiments, the Cas protein is Cas9. In some embodiments, the Cas protein is Cas12a. In some embodiments, the Cas protein is Cas12b.

In some embodiments, the Cas protein is an enzymatically dead Cas protein or a nickase. In some embodiments, the Cas protein is an enzymatically dead Cas protein. In some embodiments, the Cas protein is a nickase. In some embodiments, the Cas protein is Cas9 D10A or Cas9 H840A.

In some embodiments, the Cas nuclease, TALEN or ZFN is fused to a FokI nuclease or related nuclease domain. In some embodiments, the Cas nuclease is fused to a FokI nuclease. In some embodiments, the enzymatically dead Cas nuclease or nickase is fused to a FokI nuclease. In some embodiments, Cas9 D10A or Cas H840A is fused to a FokI nuclease. In some embodiments, a TALEN is fused to a FokI nuclease. In some embodiments, a ZFN is fused to a FokI nuclease. In some embodiments, Cas9 D10A or Cas H840A is fused to a Moloney Murine Leukemia Virus (M-MLV) reverse transcriptase.

In some embodiments, the homology arm is between about 50 to 500 nt. In some embodiments, the homology arm is about 50 nt. In some embodiments, the homology arm is about 100 nt. In some embodiments, the homology arm is about 150 nt. In some embodiments, the homology arm is about 200 nt. In some embodiments, the homology arm is about 250 nt. In some embodiments, the homology arm is about 300 nt. In some embodiments, the homology arm is about 350 nt. In some embodiments, the homology arm is about 400 nt. In some embodiments, the homology arm is about 450 nt. In some embodiments, the homology arm is about 500 nt. In some embodiments, the homology arm is greater than 500 nt.

In some embodiments, the re-editable template is between about 10-500 nt. In some embodiments, the re-editable template is about 10 nt. In some embodiments, the re-editable template is about 20 nt. In some embodiments, the re-editable template is about 30 nt. In some embodiments, the re-editable template is about 40 nt. In some embodiments, the re-editable template is about 50 nt. In some embodiments, the re-editable template is about 60 nt. In some embodiments, the re-editable template is about 70 nt. In some embodiments, the re-editable template is about 80 nt. In some embodiments, the re-editable template is about 90 nt. In some embodiments, the re-editable template is about 100 nt.

In some embodiments, the re-editable template is about 150 nt. In some embodiments, the re-editable template is about 200 nt. In some embodiments, the re-editable template is about 250 nt. In some embodiments, the re-editable template is about 300 nt. In some embodiments, the re-editable template is about 350 nt. In some embodiments, the re-editable template is about 400 nt. In some embodiments, the re-editable template is about 450 nt. In some embodiments, the re-editable template is about 500 nt.

In some embodiments, the re-editable template comprises a Protospacer Adjacent Motif (PAM). In some embodiments, the re-editable template comprises a 5′-NGG-3′ PAM and the nuclease is Cas9. In some embodiments, the re-editable template comprises a specific PAM for a specific Cas nuclease.

In some embodiments, the coding region in the genome recognizable by a guide RNA contains at least 3 mismatches relative to the sequence in the re-editable template recognizable by a guide RNA. In some embodiments, the coding region in the genome recognizable by a guide RNA contains at least 4 mismatches relative to the sequence in the re-editable template recognizable by a guide RNA. In some embodiments, off-target editing is absent.

In some embodiments, the cell is a mammalian cell. In some embodiments, the cell is a human cell. In some embodiments, the cell is a cultured cell. In some embodiments, the cell is a primary cell. In some embodiments, the cell is a non-dividing cell. In some embodiments, the cell is an immune cell. In some embodiments, the cell is a B-cell, T-cell or NK-cell. In some embodiments, the cell is a stem cell or progenitor cell. In some embodiments, the cell is an induced pluripotent stem cell (iPSC).

In some embodiments, the target locus is at least one locus selected from the group consisting of a ubiquitously expressed gene, cell division related gene, and gene with expression restricted to specific cell types. In some embodiments, the target locus is a Class I or Class II HLA gene. In some embodiments, the target locus is a Class I HLA gene. In some embodiments, the target locus is a Class II HLA gene. In some embodiments, the target locus is B2M.

In some embodiments, the gRNA that directs a double-stranded break to insert a re-editable template in a B2M exon 1 locus comprises 70%, 80%, 90%, 95%, 99% or 100% identity to GAGTAGCGCGAGCACAGCTA (SEQ ID NO: 1), AGGGTAGGAGAGACTCACGC (SEQ ID NO: 2), or GGCCGAGATGTCTCGCTCCG (SEQ ID NO: 3). In some embodiments, the gRNA that directs a double-stranded break to insert a re-editable template in a B2M exon 1 locus comprises 70% identity to any one of SEQ ID NO: 1-3. In some embodiments, the gRNA that directs a double-stranded break to insert a re-editable template in a B2M exon 1 locus comprises 75% identity to any one of SEQ ID NO: 1-3. In some embodiments, the gRNA that directs a double-stranded break to insert a re-editable template in a B2M exon 1 locus comprises 80% identity to any one of SEQ ID NO: 1-3. In some embodiments, the gRNA that directs a double-stranded break to insert a re-editable template in a B2M exon 1 locus comprises 85% identity to any one of SEQ ID NO: 1-3. In some embodiments, the gRNA that directs a double-stranded break to insert a re-editable template in a B2M exon 1 locus comprises 90% identity to any one of SEQ ID NO: 1-3. In some embodiments, the gRNA that directs a double-stranded break to insert a re-editable template in a B2M exon 1 locus comprises 95% identity to any one of SEQ ID NO: 1-3. In some embodiments, the gRNA that directs a double-stranded break to insert a re-editable template in a B2M exon 1 locus comprises 99% identity to any one of SEQ ID NO: 1-3. In some embodiments, the gRNA that directs a double-stranded break to insert a re-editable template in a B2M exon 1 locus comprises 100% identity to any one of SEQ ID NO: 1-3.

In some embodiments, the re-editable template is defined by a sequence comprising 70%, 80%, 90%, 95%, 99% or 100% identity to ATTCCTGAAGCTGACAGCATTCGGGCCGAGATGTCCAGGTCCTAATGATTAGCT GTGCTCGCCCTGCTCTCTCTGTCTGGCCTGGAGGCTATTCAGCGTGAGTCTCT CCTACCCTCCCGCTCTGGTCCTTCCTCTCCCGCTCTGCACCCTCTGTGGCCCT (SEQ ID NO: 5). In some embodiments, the re-editable template is defined by a sequence comprising 70%, identity to SEQ ID NO: 5. In some embodiments, the re-editable template is defined by a sequence comprising 75% identity to SEQ ID NO: 5. In some embodiments, the re-editable template is defined by a sequence comprising 80% identity to SEQ ID NO: 5. In some embodiments, the re-editable template is defined by a sequence comprising 85% identity to SEQ ID NO: 5. In some embodiments, the re-editable template is defined by a sequence comprising 90% identity to SEQ ID NO: 5. In some embodiments, the re-editable template is defined by a sequence comprising 95% identity to SEQ ID NO: 5. In some embodiments, the re-editable template is defined by a sequence comprising 99% identity to SEQ ID NO: 5. In some embodiments, the re-editable template is defined by a sequence comprising 100% identity to SEQ ID NO: 5.

In some embodiments, the re-editable template is defined by a sequence comprising 70%, 80%, 90%, 95%, 99% or 100% identity to AAAACGGGAAAGTCCCTCTCTCTAACCTGGCACTGCGTCGCTGGCTTGGAGACA GGTGACGGTCCCTGCGGGCCTTGTCCTGATTGGCTGGGCACGCGTTTAATATAAG TGGAGGCGTCGCGCTGGCGGGCATTCCTGAAGCTGACAGCATTCGGGCCGAGAT GTCGTAGAGCGTGTGACTAGCTGTACTGGAGCTGTGAAGCTAATCCGGTCT GGAAGCCATTCAGCGTGAGTCTCTCCTACCCTCCCGCTCTGGTCCTTCCTCTCCC GCTCTGCACCCTCTGTGGCCCTCGCTGTGCTCTCTCGCTCCGTGACTTCCCTTCTC CAAGTTCTCCTTGGT (SEQ ID NO: 6). In some embodiments, the re-editable template is defined by a sequence comprising 70%, identity to SEQ ID NO: 6. In some embodiments, the re-editable template is defined by a sequence comprising 75% identity to SEQ ID NO: 6. In some embodiments, the re-editable template is defined by a sequence comprising 80% identity to SEQ ID NO: 6. In some embodiments, the re-editable template is defined by a sequence comprising 85% identity to SEQ ID NO: 6. In some embodiments, the re-editable template is defined by a sequence comprising 90% identity to SEQ ID NO: 6. In some embodiments, the re-editable template is defined by a sequence comprising 95% identity to SEQ ID NO: 6. In some embodiments, the re-editable template is defined by a sequence comprising 99% identity to SEQ ID NO: 6. In some embodiments, the re-editable template is defined by a sequence comprising 100% identity to SEQ ID NO: 6.

In some embodiments, the gRNA that directs a double-stranded break to insert a re-editable template in a B2M exon 2 locus comprises 70%, 80%, 90%, 95%, 99% or 100% identity to AAGTCAACTTCAATGTCGGA (SEQ ID NO: 7), AGTCACATGGTTCACACGGC (SEQ ID NO: 8), or ACTTGTCTTTCAGCAAGGAC (SEQ ID NO: 9). In some embodiments, the gRNA that directs a double-stranded break to insert a re-editable template in a B2M exon 2 locus comprises 70% identity to any one of SEQ ID NO: 7-9. In some embodiments, the gRNA that directs a double-stranded break to insert a re-editable template in a B2M exon 2 locus comprises 75% identity to any one of SEQ ID NO: 7-9. In some embodiments, the gRNA that directs a double-stranded break to insert a re-editable template in a B2M exon 2 locus comprises 80% identity to any one of SEQ ID NO: 7-9. In some embodiments, the gRNA that directs a double-stranded break to insert a re-editable template in a B2M exon 2 locus comprises 85% identity to any one of SEQ ID NO: 7-9. In some embodiments, the gRNA that directs a double-stranded break to insert a re-editable template in a B2M exon 2 locus comprises 90% identity to any one of SEQ ID NO: 7-9. In some embodiments, the gRNA that directs a double-stranded break to insert a re-editable template in a B2M exon 2 locus comprises 95% identity to any one of SEQ ID NO: 7-9. In some embodiments, the gRNA that directs a double-stranded break to insert a re-editable template in a B2M exon 2 locus comprises 99% identity to any one of SEQ ID NO: 7-9. In some embodiments, the gRNA that directs a double-stranded break to insert a re-editable template in a B2M exon 2 locus comprises 100% identity to any one of SEQ ID NO: 7-9.

In some embodiments, the gRNA that recognizes the re-editable template inserted in a B2M exon 1 locus comprises 70%, 80%, 90%, 95%, 99% or 100% identity to GAGCGTGTGACTAGCTGTAC (SEQ ID NO: 4). In some embodiments, the gRNA that recognizes the re-editable template inserted in a B2M exon 1 locus comprises 70% identity to GAGCGTGTGACTAGCTGTAC (SEQ ID NO: 4). In some embodiments, the gRNA that recognizes the re-editable template inserted in a B2M exon 1 locus comprises 80% identity to GAGCGTGTGACTAGCTGTAC (SEQ ID NO: 4). In some embodiments, the gRNA that recognizes the re-editable template inserted in a B2M exon 1 locus comprises 90% identity to GAGCGTGTGACTAGCTGTAC (SEQ ID NO: 4). In some embodiments, the gRNA that recognizes the re-editable template inserted in a B2M exon 1 locus comprises 95% identity to GAGCGTGTGACTAGCTGTAC (SEQ ID NO: 4). In some embodiments, the gRNA that recognizes the re-editable template inserted in a B2M exon 1 locus comprises 99% identity to GAGCGTGTGACTAGCTGTAC (SEQ ID NO: 4). In some embodiments, the gRNA that recognizes the re-editable template inserted in a B2M exon 1 locus comprises 99% identity to GAGCGTGTGACTAGCTGTAC (SEQ ID NO: 4). In some embodiments, the gRNA that recognizes the re-editable template inserted in a B2M exon 1 locus comprises 100% identity to GAGCGTGTGACTAGCTGTAC (SEQ ID NO: 4).

In some embodiments, the gRNA that recognizes the re-editable template inserted in a B2M exon 2 locus comprises 70%, 80%, 90%, 95%, 99% or 100% identity to CCTAGATCCAATAGTAGAGT (SEQ ID NO: 10) or GGTCACGTGGTTCACCCTAC (SEQ ID NO: 11). In some embodiments, the gRNA that recognizes the re-editable template inserted in a B2M exon 2 locus comprises 70% identity to any one of SEQ ID NO: 10 or 11. In some embodiments, the gRNA that recognizes the re-editable template inserted in a B2M exon 2 locus comprises 75% identity to any one of SEQ ID NO: 10 or 11. In some embodiments, the gRNA that recognizes the re-editable template inserted in a B2M exon 2 locus comprises 80% identity to any one of SEQ ID NO: 10 or 11. In some embodiments, the gRNA that recognizes the re-editable template inserted in a B2M exon 2 locus comprises 85% identity to any one of SEQ ID NO: 10 or 11. In some embodiments, the gRNA that recognizes the re-editable template inserted in a B2M exon 2 locus comprises 90% identity to any one of SEQ ID NO: 10 or 11. In some embodiments, the gRNA that recognizes the re-editable template inserted in a B2M exon 2 locus comprises 95% identity to any one of SEQ ID NO: 10 or 11. In some embodiments, the gRNA that recognizes the re-editable template inserted in a B2M exon 2 locus comprises 99% identity to any one of SEQ ID NO: 10 or 11. In some embodiments, the gRNA that recognizes the re-editable template inserted in a B2M exon 2 locus comprises 100% identity to any one of SEQ ID NO: 10 or 11. In some embodiments, the gRNA that recognizes the re-editable template inserted in a B2M exon 1 locus comprises 70%, 80%, 90%, 95%, 99% or 100% identity to GAGCGTGTGACTAGCTGTAC (SEQ ID NO: 4). In some embodiments, the gRNA that recognizes the re-editable template inserted in a B2M exon 1 locus comprises 70% identity to GAGCGTGTGACTAGCTGTAC (SEQ ID NO: 4). In some embodiments, the gRNA that recognizes the re-editable template inserted in a B2M exon 1 locus comprises 80% identity to GAGCGTGTGACTAGCTGTAC (SEQ ID NO: 4). In some embodiments, the gRNA that recognizes the re-editable template inserted in a B2M exon 1 locus comprises 90% identity to GAGCGTGTGACTAGCTGTAC (SEQ ID NO: 4). In some embodiments, the gRNA that recognizes the re-editable template inserted in a B2M exon 1 locus comprises 95% identity to GAGCGTGTGACTAGCTGTAC (SEQ ID NO: 4). In some embodiments, the gRNA that recognizes the re-editable template inserted in a B2M exon 1 locus comprises 99% identity to GAGCGTGTGACTAGCTGTAC (SEQ ID NO: 4). In some embodiments, the gRNA that recognizes the re-editable template inserted in a B2M exon 1 locus comprises 100% identity to GAGCGTGTGACTAGCTGTAC (SEQ ID NO: 4).

In some embodiments, the re-editable template is defined by a sequence comprising 70%, 80%, 90%, 95%, 99% or 100% identity to AAATGTAAACACTTGGTGCCTGATATAGCTTGACACCAAGTTAGCCCCAAGTGA AATACCCTGGCAATATTAATGTGTCTTTTCCCGATATTCCTCAGGTACCCCCTAG ATCCAATAGTAGAGTAGGTGACCAGCCTAGAACGGAGCCTGTAGGGTGAAC CACGTGACCCTGTAACAGTGGGGTAAGTCTTACATTCTTTTGTAAGCTGCTGAA AGTTGTGTATGAGTAGTCATATCATAAAGCTGCTTTGATATAAAAAAGGTCTATG GCCATACTACCC (SEQ ID NO: 12). In some embodiments, the re-editable template is defined by a sequence comprising 70% identity to SEQ ID NO: 12. In some embodiments, the re-editable template is defined by a sequence comprising 75% identity to SEQ ID NO: 12. In some embodiments, the re-editable template is defined by a sequence comprising 80% identity to SEQ ID NO: 12. In some embodiments, the re-editable template is defined by a sequence comprising 85% identity to SEQ ID NO: 12. In some embodiments, the re-editable template is defined by a sequence comprising 90% identity to SEQ ID NO: 12. In some embodiments, the re-editable template is defined by a sequence comprising 95% identity to SEQ ID NO: 12. In some embodiments, the re-editable template is defined by a sequence comprising 99% identity to SEQ ID NO: 12. In some embodiments, the re-editable template is defined by a sequence comprising 100% identity to SEQ ID NO: 12.

In some embodiments, the target locus is CIITA.

In some embodiments, the gRNA that directs a double-stranded break to insert a re-editable template in exon 2 of the CIITA locus comprises 70%, 80%, 90%, 95%, 99% or 100% identity to ATGGAGTTGGGGCCCCTAGA (SEQ ID NO. 13), CTACCACTTCTATGACCAGA (SEQ ID NO: 14) or GTGGCACACTGTGAGCTGCC (SEQ ID NO: 15). In some embodiments, the gRNA that directs a double-stranded break to insert a re-editable template in exon 2 of the CIITA locus comprises 70% identity to any one of SEQ ID NO: 13-15. In some embodiments, the gRNA that directs a double-stranded break to insert a re-editable template in exon 2 of the CIITA locus comprises 75% identity to any one of SEQ ID NO: 13-15. In some embodiments, the gRNA that directs a double-stranded break to insert a re-editable template in exon 2 of the CIITA locus comprises 80% identity to any one of SEQ ID NO: 13-15. In some embodiments, the gRNA that directs a double-stranded break to insert a re-editable template in exon 2 of the CIITA locus comprises 85% identity to any one of SEQ ID NO: 13-15. In some embodiments, the gRNA that directs a double-stranded break to insert a re-editable template in exon 2 of the CIITA locus comprises 90% identity to any one of SEQ ID NO: 13-15. In some embodiments, the gRNA that directs a double-stranded break to insert a re-editable template in exon 2 of the CIITA locus comprises 95% identity to any one of SEQ ID NO: 13-15. In some embodiments, the gRNA that directs a double-stranded break to insert a re-editable template in exon 2 of the CIITA locus comprises 99% identity to any one of SEQ ID NO: 13-15. In some embodiments, the gRNA that directs a double-stranded break to insert a re-editable template in exon 2 of the CIITA locus comprises 100% identity to any one of SEQ ID NO: 13-15.

In some embodiments, the re-editable template is defined by a sequence comprising 70%, 80%, 90%, 95%, 99% or 100% identity to TTCTGCCTCTTTCCAACACCCTGTGAGGTGACTGAGCATTGTCTTCCCTCCCAGGC AGTTCCCAGTGTGCTACCATGGAGTTGTGACCCCTATAATGAGACCTGGCTGG AGAAGAAGAGATTGAGCTCTACTCAGGTGGGCCCTCCTCC (SEQ ID NO: 18). In some embodiments, the re-editable template is defined by a sequence comprising 70% identity to
SEQ ID NO: 18. In some embodiments, the re-editable template is defined by a sequence comprising 75% identity to SEQ ID NO: 18. In some embodiments, the re-editable template is defined by a sequence comprising 80% identity to SEQ ID NO: 18. In some embodiments, the re-editable template is defined by a sequence comprising 85% identity to SEQ ID NO: 18. In some embodiments, the re-editable template is defined by a sequence comprising 90% identity to SEQ ID NO: 18. In some embodiments, the re-editable template is defined by a sequence comprising 90% identity to SEQ ID NO: 18. In some embodiments, the re-editable template is defined by a sequence comprising 95% identity to SEQ ID NO: 18. In some embodiments, the re-editable template is defined by a sequence comprising 99% identity to SEQ ID NO: 18. In some embodiments, the re-editable template is defined by a sequence comprising 100% identity to SEQ ID NO: 18.

In some embodiments, the gRNA that recognizes the re-editable template inserted in a CIITA exon 2 locus comprises 70%, 80%, 90%, 95%, 99% or 100% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 70% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 80% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 90% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 95% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 99% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 100% identity to GTGACCCCTATAATGAGACC (SEQ ID NO: 16) or CAGTTCCCAGTGTGCTACCA (SEQ ID NO: 17).

In some embodiments, the gRNA that directs a double-stranded break to insert a re-editable template in exon 3 of the CIITA locus comprises 70%, 80%, 90%, 95%, 99% or 100% identity to AGGCTGTTGTGTGACATGGA (SEQ ID NO: 19). In some embodiments, the gRNA that directs a double-stranded break to insert a re-editable template in exon 3 of the CIITA locus comprises 70% identity to SEQ ID NO: 19. In some embodiments, the gRNA that directs a double-stranded break to insert a re-editable template in exon 3 of the CIITA locus comprises 75% identity to SEQ ID NO: 19. In some embodiments, the gRNA that directs a double-stranded break to insert a re-editable template in exon 3 of the CIITA locus comprises 80% identity to SEQ ID NO: 19. In some embodiments, the gRNA that directs a double-stranded break to insert a re-editable template in exon 3 of the CIITA locus comprises 85% identity to SEQ ID NO: 19. In some embodiments, the gRNA that directs a double-stranded break to insert a re-editable template in exon 3 of the CIITA locus comprises 90% identity to SEQ ID NO: 19. In some embodiments, the gRNA that directs a double-stranded break to insert a re-editable template in exon 3 of the CIITA locus comprises 95% identity to SEQ ID NO: 19. In some embodiments, the gRNA that directs a double-stranded break to insert a re-editable template in exon 3 of the CIITA locus comprises 99% identity to SEQ ID NO: 19. In some embodiments, the gRNA that directs a double-stranded break to insert a re-editable template in exon 3 of the CIITA locus comprises 100% identity to SEQ ID NO: 19.

In some embodiments, the gRNA that recognizes the re-editable template inserted in exon 3 of the CIITA locus comprises 70%, 80%, 90%, 95%, 99% or 100% identity TGACTGATGTAAGACTAGTA (SEQ ID NO: 20). In some embodiments, the gRNA that recognizes the re-editable template inserted in exon 3 of the CIITA locus comprises 70% identity to SEQ ID NO: 20. In some embodiments, the gRNA that recognizes the re-editable template inserted in exon 3 of the CIITA locus comprises 75% identity to SEQ ID NO: 20. In some embodiments, the gRNA that recognizes the re-editable template inserted in exon 3 of the CIITA locus comprises 80% identity to SEQ ID NO: 20. In some embodiments, the gRNA that recognizes the re-editable template inserted in exon 3 of the CIITA locus comprises 85% identity to SEQ ID NO: 20. In some embodiments, the gRNA that recognizes the re-editable template inserted in exon 3 of the CIITA locus comprises 90% identity to SEQ ID NO: 20. In some embodiments, the gRNA that recognizes the re-editable template inserted in exon 3 of the CIITA locus comprises 95% identity to SEQ ID NO: 20. In some embodiments, the gRNA that recognizes the re-editable template inserted in exon 3 of the CIITA locus comprises 99% identity to SEQ ID NO: 20. In some embodiments, the gRNA that recognizes the re-editable template inserted in exon 3 of the CIITA locus comprises 100% identity to SEQ ID NO: 20.

In some embodiments, the re-editable template is defined by a sequence comprising 70%, 80%, 90%, 95%, 99% or 100% identity to AAATTTCCTTCTTCATCCAAGGGACTTTTCCTCCCAGAACCCGACACAGACACCA TCAACTGCGACCAGTTCAGCTGACTGATGTAAGACTAGTAAGGTGATGAAGAG ACCAGGGAGGCTTATGCCAATATCGGTGAGGAAGCACCTGAGCCCAGAAAAGGA CAATCAAGGGCAAGAGTTCTTTGCTGCCACTTGTCA (SEQ ID NO: 21). In some embodiments, the re-editable template is defined by a sequence comprising 70% identity to SEQ ID NO: 21. In some embodiments, the re-editable template is defined by a sequence comprising 75% identity to SEQ ID NO: 21. In some embodiments, the re-editable template is defined by a sequence comprising 80% identity to SEQ ID NO: 21. In some embodiments, the re-editable template is defined by a sequence comprising 85% identity to SEQ ID NO: 21. In some embodiments, the re-editable template is defined by a sequence comprising 90% identity to SEQ ID NO: 21. In some embodiments, the re-editable template is defined by a sequence comprising 95% identity to SEQ ID NO: 21. In some embodiments, the re-editable template is defined by a sequence comprising 99% identity to SEQ ID NO: 21. In some embodiments, the re-editable template is defined by a sequence comprising 100% identity to SEQ ID NO: 21.

In some embodiments, the re-editable template comprises an exogenous protein-coding gene. In some embodiments, the exogenous gene is an immune modulatory or cloaking gene. In some embodiments, the exogenous gene is an immune modulatory gene. In some embodiments, the exogenous gene is a cloaking gene. In some embodiments, the exogenous gene expresses a protein that leads to cell death, wherein the gene is selected from the group consisting of HSV-TK, iCaspase8 and iCaspase9. In some embodiments, the exogenous gene expresses a protein that leads to cell death of proliferating cells in the presence of a specific compound, wherein the gene is HSV-TK. In some embodiments, the exogenous gene expresses a protein that after activation leads to cell death, wherein the gene is iCaspase8. In some embodiments, the exogenous gene expresses a protein that leads after activation to cell death, wherein the gene is iCaspase9.

In some embodiments, the re-editable template comprises a chimeric antigen receptor (CAR) gene.

In some embodiments, provided is an isolated cell engineered by the method provided herein.

In some embodiments, provided herein is an engineered cell comprising a re-editable target locus, wherein the re-editable target locus comprises an exogenous re-editable template sequence with no substantial sequence similarity to any region in the rest of the genome, and wherein the re-editable template sequence is recognizable by a genome editing system.

In some embodiments, the genome editing system is a CRISPR-Cas-related nuclease system, a Transcription Activator-Like Effector Nuclease (TALEN) system, or a Zinc-Finger Nuclease (ZFN) system. In some embodiments, the genome editing system is a CRISPR-Cas-related nuclease system. In some embodiments, the genome editing system is a Transcription Activator-Like Effector Nuclease (TALEN) system. In some embodiments, the genome editing system is a Zinc-Finger Nuclease (ZFN) system.

In some embodiments, the cell comprises a Cas protein, wherein the Cas protein is Cas9, Cas12a or Cas12b. In some embodiments, the Cas protein is Cas9. In some embodiments, the Cas protein is Cas12a. In some embodiments, the Cas protein is Cas12b.

In some embodiments, the Cas protein of the engineered cell is an enzymatically dead Cas protein or a nickase. In some embodiments, the Cas protein is an enzymatically dead Cas protein. In some embodiments, the Cas protein is a nickase. In some embodiments, the Cas protein is Cas9 D10A or Cas9 H840A.

In some embodiments, the Cas protein, TALEN or ZFN is fused to a FokI nuclease or related nuclease domain. In some embodiments, the Cas protein is fused to a FokI nuclease. In some embodiments, the enzymatically dead Cas protein or nickase is fused to a FokI nuclease. In some embodiments, Cas9 D10A or Cas9 H840A is fused to a FokI nuclease or M-MLV reverse transcriptase. In some embodiments, a TALEN is fused to a FokI nuclease. In some embodiments, a ZFN is fused to a FokI nuclease.

In some embodiments, the homology arm is between about 50 to 500 nt. In some embodiments, the homology arm is about 50 nt. In some embodiments, the homology arm is about 100 nt. In some embodiments, the homology arm is about 150 nt. In some embodiments, the homology arm is about 200 nt. In some embodiments, the homology arm is about 250 nt. In some embodiments, the homology arm is about 300 nt. In some embodiments, the homology arm is about 350 nt. In some embodiments, the homology arm is about 400 nt. In some embodiments, the homology arm is about 450 nt. In some embodiments, the homology arm is about 500 nt. In some embodiments, the homology arm is greater than about 500 nt.

In some embodiments, the re-editable template is between about 10-500 nt. In some embodiments, the re-editable template is about 10 nt. In some embodiments, the re-editable template is about 20 nt. In some embodiments, the re-editable template is about 30 nt. In some embodiments, the re-editable template is about 40 nt. In some embodiments, the re-editable template is about 50 nt. In some embodiments, the re-editable template is about 60 nt. In some embodiments, the re-editable template is about 70 nt. In some embodiments, the re-editable template is about 80 nt. In some embodiments, the re-editable template is about 90 nt. In some embodiments, the re-editable template is about 100 nt.

In some embodiments, the re-editable template is about 150 nt. In some embodiments, the re-editable template is about 200 nt. In some embodiments, the re-editable template is about 250 nt. In some embodiments, the re-editable template is about 300 nt. In some embodiments, the re-editable template is about 350 nt. In some embodiments, the re-editable template is about 400 nt. In some embodiments, the re-editable template is about 450 nt. In some embodiments, the re-editable template is about 500 nt.

In some embodiments, the re-editable template of the cell comprises a Protospacer Adjacent Motif (PAM). In some embodiments, the re-editable template comprises a 5′-NGG-3′ PAM and the nuclease is Cas9. In some embodiments, the re-editable template comprises a specific PAM for a specific Cas nuclease.

In some embodiments, the coding region in the genome recognizable by a guide RNA contains at least 3 mismatches. In some embodiments, the coding region in the genome recognizable by a guide RNA contains 3 mismatches. In some embodiments, the coding region in the genome recognizable by a guide RNA contains greater than 3 mismatches. In some embodiments, the coding region in the genome recognizable by a guide RNA contains 4 mismatches. In some embodiments, the coding region in the genome recognizable by a guide RNA contains 5 mismatches. In some embodiments, the coding region in the genome recognizable by a guide RNA contains 6 mismatches. In some embodiments, the coding region in the genome recognizable by a guide RNA contains 7 mismatches. In some embodiments, the coding region in the genome recognizable by a guide RNA contains 8 mismatches. In some embodiments, the coding region in the genome recognizable by a guide RNA contains 9 mismatches. In some embodiments, the coding region in the genome recognizable by a guide RNA contains 10 mismatches. In some embodiments, off-target editing is absent.

In some embodiments, the cell is a mammalian cell. In some embodiments, the cell is a human cell. In some embodiments, the cell is a cultured cell. In some embodiments, the cell is a primary cell. In some embodiments, the cell is a non-dividing cell. In some embodiments, the cell is an immune cell. In some embodiments, the cell is a B-cell, T-cell or NK-cell. In some embodiments, the cell is a B-cell. In some embodiments, the cell is a T-cell. In some embodiments, the cell is a NK-cell. In some embodiments, the cell is a stem cell or progenitor cell. In some embodiments, the cell is a stem cell. In some embodiments, the cell is a progenitor cell. In some embodiments, the cell is an induced pluripotent stem cell (iPSC).

In some embodiments, the target locus is an immune gene. In some embodiments, the target locus is a Class I or Class II HLA gene. In some embodiments, the target locus is a Class I HLA gene. In some embodiments, the target locus is a Class II HLA gene. In some embodiments, the target locus is B2M.

In some embodiments, the gRNA in the cell that directs a double-stranded break to insert a re-editable template in a B2M exon 1 locus comprises 70%, 80%, 90%, 95%, 99% or 100% identity to GAGTAGCGCGAGCACAGCTA (SEQ ID NO: 1), AGGGTAGGAGAGACTCACGC (SEQ ID NO: 2) or GGCCGAGATGTCTCGCTCCG (SEQ ID NO: 3). In some embodiments, the gRNA in the cell that directs a double-stranded break to insert a re-editable template in a B2M exon 1 locus comprises 70% identity to any one of SEQ ID NO: 1-3. In some embodiments, the re-editable template in the cell is defined by a sequence comprising 75% identity to any one of SEQ ID NO: 1-3. In some embodiments, the gRNA in the cell that directs a double-stranded break to insert a re-editable template in a B2M exon 1 locus comprises 80% identity to any one of SEQ ID NO: 1-3. In some embodiments, the re-editable template in the cell is defined by a sequence comprising 85% identity to any one of SEQ ID NO: 1-3. In some embodiments, the gRNA in the cell that directs a double-stranded break to insert a re-editable template in a B2M exon 1 locus comprises 90% identity to any one of SEQ ID NO: 1-3. In some embodiments, the gRNA in the cell that directs a double-stranded break to insert a re-editable template in a B2M exon 1 locus comprises 95% identity to any one of SEQ ID NO: 1-3. In some embodiments, the gRNA in the cell that directs a double-stranded break to insert a re-editable template in a B2M exon 1 locus comprises 99% identity to any one of SEQ ID NO: 1-3. In some embodiments, the gRNA in the cell that directs a double-stranded break to insert a re-editable template in a B2M exon 1 locus comprises 100% identity to any one of SEQ ID NO: 1-3.

In some embodiments, the re-editable template in the cell is defined by a sequence comprising 70%, 80%, 90%, 95%, 99% or 100% identity to ATTCCTGAAGCTGACAGCATTCGGGCCGAGATGTCCAGGTCCTAATGATTAGCT GTGCTCGCCCTGCTCTCTCTGTCTGGCCTGGAGGCTATTCAGCGTGAGTCTCT CCTACCCTCCCGCTCTGGTCCTTCCTCTCCCGCTCTGCACCCTCTGTGGCCCT (SEQ ID NO: 5). In some embodiments, the re-editable template in the cell is defined by a sequence comprising 70% identity to SEQ ID NO: 5. In some embodiments, the re-editable template in the cell is defined by a sequence comprising 75% identity to SEQ ID NO: 5. In some embodiments, the re-editable template in the cell is defined by a sequence comprising 80% identity to SEQ ID NO: 5. In some embodiments, the re-editable template in the cell is defined by a sequence comprising 85% identity to SEQ ID NO: 5. In some embodiments, the re-editable template in the cell is defined by a sequence comprising 90% identity to SEQ ID NO: 5. In some embodiments, the re-editable template in the cell is defined by a sequence comprising 95% identity to SEQ ID NO: 5. In some embodiments, the re-editable template in the cell is defined by a sequence comprising 99% identity to SEQ ID NO: 5. In some embodiments, the re-editable template in the cell is defined by a sequence comprising 100% identity to SEQ ID NO: 5.

In some embodiments, the re-editable template in the cell is codon-optimized. In some embodiments, the re-editable template in the cell is defined by a sequence comprising 70%, 80%, 90%, 95%, 99% or 100% identity to AAAACGGGAAAGTCCCTCTCTCTAACCTGGCACTGCGTCGCTGGCTTGGAGACA GGTGACGGTCCCTGCGGGCCTTGTCCTGATTGGCTGGGCACGCGTTTAATATAAG TGGAGGCGTCGCGCTGGCGGGCATTCCTGAAGCTGACAGCATTCGGGCCGAGAT GTCGTAGAGCGTGTGACTAGCTGTACTGGAGCTGTGAAGCTAATCCGGTCT GGAAGCCATTCAGCGTGAGTCTCTCCTACCCTCCCGCTCTGGTCCTTCCTCTCCC GCTCTGCACCCTCTGTGGCCCTCGCTGTGCTCTCTCGCTCCGTGACTTCCCTTCTC CAAGTTCTCCTTGGT (SEQ ID NO: 6). In some embodiments, the re-editable template in the cell is defined by a sequence comprising 70% identity to SEQ ID NO: 6. In some embodiments, the re-editable template in the cell is defined by a sequence comprising 75% identity to SEQ ID NO: 6. In some embodiments, the re-editable template in the cell is defined by a sequence comprising 80% identity to SEQ ID NO: 6. In some embodiments, the re-editable template in the cell is defined by a sequence comprising 85% identity to SEQ ID NO: 6. In some embodiments, the re-editable template in the cell is defined by a sequence comprising 90% identity to SEQ ID NO: 6. In some embodiments, the re-editable template in the cell is defined by a sequence comprising 95% identity to SEQ ID NO: 6. In some embodiments, the re-editable template in the cell is defined by a sequence comprising 99% identity to SEQ ID NO: 6. In some embodiments, the re-editable template in the cell is defined by a sequence comprising 100% identity to SEQ ID NO: 6.

In some embodiments, the gRNA that recognizes the re-editable template inserted in a B2M exon 1 locus comprises 70%, 80%, 90%, 95%, 99% or 100% identity to GAGCGTGTGACTAGCTGTAC (SEQ ID NO: 4). In some embodiments, the gRNA that recognizes the re-editable template inserted in a B2M exon 1 locus comprises 70% identity to GAGCGTGTGACTAGCTGTAC (SEQ ID NO: 4). In some embodiments, the gRNA that recognizes the re-editable template inserted in a B2M exon 1 locus comprises 80% identity to GAGCGTGTGACTAGCTGTAC (SEQ ID NO: 4). In some embodiments, the gRNA that recognizes the re-editable template inserted in a B2M exon 1 locus comprises 90% identity to GAGCGTGTGACTAGCTGTAC (SEQ ID NO: 4). In some embodiments, the gRNA that recognizes the re-editable template inserted in a B2M exon 1 locus comprises 95% identity to GAGCGTGTGACTAGCTGTAC (SEQ ID NO: 4). In some embodiments, the gRNA that recognizes the re-editable template inserted in a B2M exon 1 locus comprises 100% identity to GAGCGTGTGACTAGCTGTAC (SEQ ID NO: 4).

In some embodiments, the gRNA in the cell that directs a double-stranded break to insert a re-editable template in a B2M exon 2 locus comprises 70%, 80%, 90%, 95%, 99% or 100% identity to AAGTCAACTTCAATGTCGGA (SEQ ID NO: 7), AGTCACATGGTTCACACGGC (SEQ ID NO: 8), or ACTTGTCTTTCAGCAAGGAC (SEQ ID NO: 9). In some embodiments, the gRNA in the cell that directs a double-stranded break to insert a re-editable template in a B2M exon 2 locus comprises 70% identity to any one of SEQ ID NO: 7-9. In some embodiments, the gRNA in the cell that directs a double-stranded break to insert a re-editable template in a B2M exon 2 locus comprises 75% identity to any one of SEQ ID NO: 7-9. In some embodiments, the gRNA in the cell that directs a double-stranded break to insert a re-editable template in a B2M exon 2 locus comprises 80% identity to any one of SEQ ID NO: 7-9. In some embodiments, the gRNA in the cell that directs a double-stranded break to insert a re-editable template in a B2M exon 2 locus comprises 85% identity to any one of SEQ ID NO: 7-9. In some embodiments, the gRNA in the cell that directs a double-stranded break to insert a re-editable template in a B2M exon 2 locus comprises 90% identity to any one of SEQ ID NO: 7-9. In some embodiments, the gRNA in the cell that directs a double-stranded break to insert a re-editable template in a B2M exon 2 locus comprises 95% identity to any one of SEQ ID NO: 7-9. In some embodiments, the gRNA in the cell that directs a double-stranded break to insert a re-editable template in a B2M exon 2 locus comprises 99% identity to any one of SEQ ID NO: 7-9. In some embodiments, the gRNA in the cell that directs a double-stranded break to insert a re-editable template in a B2M exon 2 locus comprises 100% identity to any one of SEQ ID NO: 7-9.

In some embodiments, the gRNA in the cell that recognizes the re-editable template inserted in a B2M exon 2 locus comprises 70%, 80%, 90%, 95%, 99% or 100% identity to CCTAGATCCAATAGTAGAGT (SEQ ID NO: 10) or GGTCACGTGGTTCACCCTAC (SEQ ID NO: 11). In some embodiments, the gRNA in the cell that recognizes the re-editable template inserted in a B2M exon 2 locus comprises 70% identity to SEQ ID NO: 10 or 11. In some embodiments, the gRNA in the cell that recognizes the re-editable template inserted in a B2M exon 2 locus comprises 75% identity to SEQ ID NO: 10 or 11. In some embodiments, the gRNA in the cell that recognizes the re-editable template inserted in a B2M exon 2 locus comprises 80% identity to SEQ ID NO: 10 or 11. In some embodiments, the gRNA in the cell that recognizes the re-editable template inserted in a B2M exon 2 locus comprises 85% identity to SEQ ID NO: 10 or 11. In some embodiments, the gRNA in the cell that recognizes the re-editable template inserted in a B2M exon 2 locus comprises 90% identity to SEQ ID NO: 10 or 11. In some embodiments, the gRNA in the cell that recognizes the re-editable template inserted in a B2M exon 2 locus comprises 95% identity to SEQ ID NO: 10 or 11. In some embodiments, the gRNA in the cell that recognizes the re-editable template inserted in a B2M exon 2 locus comprises 99% identity to SEQ ID NO: 10 or 11. In some embodiments, the gRNA in the cell that recognizes the re-editable template inserted in a B2M exon 2 locus comprises 100% identity to SEQ ID NO: 10 or 11.

In some embodiments, the re-editable template in the cell is defined by a sequence comprising 70%, 80%, 90%, 95%, 99% or 100% identity to AAATGTAAACACTTGGTGCCTGATATAGCTTGACACCAAGTTAGCCCCAAGTGA AATACCCTGGCAATATTAATGTGTCTTTTCCCGATATTCCTCAGGTACCCCCTAG ATCCAATAGTAGAGTAGGTGACCAGCCTAGAACGGAGCCTGTAGGGTGAAC CACGTGACCCTGTAACAGTGGGGTAAGTCTTACATTCTTTTGTAAGCTGCTGAA AGTTGTGTATGAGTAGTCATATCATAAAGCTGCTTTGATATAAAAAAGGTCTATG GCCATACTACCC (SEQ ID NO: 12). In some embodiments, the re-editable template in the cell is defined by a sequence comprising 70% identity to SEQ ID NO: 12. In some embodiments, the re-editable template in the cell is defined by a sequence comprising 75% identity to SEQ ID NO: 12. In some embodiments, the re-editable template in the cell is defined by a sequence comprising 80% identity to SEQ ID NO: 12. In some embodiments, the re-editable template in the cell is defined by a sequence comprising 85% identity to SEQ ID NO: 12. In some embodiments, the re-editable template in the cell is defined by a sequence comprising 90% identity to SEQ ID NO: 12. In some embodiments, the re-editable template in the cell is defined by a sequence comprising 95% identity to SEQ ID NO: 12. In some embodiments, the re-editable template in the cell is defined by a sequence comprising 99% identity to SEQ ID NO: 12. In some embodiments, the re-editable template in the cell is defined by a sequence comprising 100% identity to SEQ ID NO: 12.

In some embodiments, the target locus is CIITA.

In some embodiments, the gRNA in the cell that directs a double-stranded break to insert a re-editable template in exon 2 of the CIITA locus comprises 70%, 80%, 90%, 95%, 99% or 100% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15). In some embodiments, the gRNA in the cell that directs a double-stranded break to insert a re-editable template in exon 2 of the CIITA locus comprises 70% identity to any one of SEQ ID NO: 13-15 In some embodiments, the gRNA in the cell that directs a double-stranded break to insert a re-editable template in exon 2 of the CIITA locus comprises 75% identity to any one of SEQ ID NO: 13-15. In some embodiments, the gRNA in the cell that directs a double-stranded break to insert a re-editable template in exon 2 of the CIITA locus comprises 80% identity to any one of SEQ ID NO: 13-15. In some embodiments, the gRNA in the cell that directs a double-stranded break to insert a re-editable template in exon 2 of the CIITA locus comprises 85% identity to any one of SEQ ID NO: 13-15. In some embodiments, the gRNA in the cell that directs a double-stranded break to insert a re-editable template in exon 2 of the CIITA locus comprises 90% identity to any one of SEQ ID NO: 13-15. In some embodiments, the gRNA in the cell that directs a double-stranded break to insert a re-editable template in exon 2 of the CIITA locus comprises 95% identity to any one of SEQ ID NO: 13-15. In some embodiments, the gRNA in the cell that directs a double-stranded break to insert a re-editable template in exon 2 of the CIITA locus comprises 100% identity to any one of SEQ ID NO: 13-15.

In some embodiments, the re-editable template in the cell is defined by a sequence comprising 70%, 80%, 90%, 95%, 99% or 100% identity to TTCTGCCTCTTTCCAACACCCTGTGAGGTGACTGAGCATTGTCTTCCCTCCCAGGC AGTTCCCAGTGTGCTACCATGGAGTTGTGACCCCTATAATGAGACCTGGCTGG AGAAGAAGAGATTGAGCTCTACTCAGGTGGGCCCTCCTCC (SEQ ID NO: 18). In some embodiments, the re-editable template in the cell is defined by a sequence comprising 70% identity to SEQ ID NO: 18. In some embodiments, the re-editable template in the cell is defined by a sequence comprising 75% identity to SEQ ID NO: 18. In some embodiments, the re-editable template in the cell is defined by a sequence comprising 80% identity to SEQ ID NO: 18. In some embodiments, the re-editable template in the cell is defined by a sequence comprising 85% identity to SEQ ID NO: 18. In some embodiments, the re-editable template in the cell is defined by a sequence comprising 90% identity to SEQ ID NO: 18. In some embodiments, the re-editable template in the cell is defined by a sequence comprising 95% identity to SEQ ID NO: 18. In some embodiments, the re-editable template in the cell is defined by a sequence comprising 99% identity to SEQ ID NO: 18. In some embodiments, the re-editable template in the cell is defined by a sequence comprising 100% identity to SEQ ID NO: 18.

In some embodiments, the gRNA that recognizes the re-editable template inserted in a CIITA exon 2 locus comprises 70%, 80%, 90%, 95%, 99% or 100% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 70% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 80% identity to GTGACCCCTATAATGAGACC (SEQ ID NO: 16) or CAGTTCCCAGTGTGCTACCA

(SEQ ID NO: 17). In some embodiments, the gRNA that recognizes the re-editable template inserted in a CIITA exon 2 locus comprises 90% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 95% identity to GTGACCCCTATAATGAGACC (SEQ ID NO: 16) or CAGTTCCCAGTGTGCTACCA (SEQ ID NO: 17) In some embodiments, the gRNA that recognizes the re-editable template inserted in a CIITA exon 2 locus comprises 100% identity to GTGACCCCTATAATGAGACC (SEQ ID NO: 16) or CAGTTCCCAGTGTGCTACCA (SEQ ID NO: 17).

In some embodiments, the gRNA that directs a double-stranded break to insert a re-editable template in exon 3 of the CIITA locus comprises 70%, 80%, 90%, 95%, 99% or 100% identity to AGGCTGTTGTGTGACATGGA (SEQ ID NO: 19). In some embodiments, the gRNA that directs a double-stranded break to insert a re-editable template in exon 3 of the CIITA locus comprises 70% identity to SEQ ID NO: 19. In some embodiments, the gRNA that directs a double-stranded break to insert a re-editable template in exon 3 of the CIITA locus comprises 75% identity to SEQ ID NO: 19. In some embodiments, the gRNA that directs a double-stranded break to insert a re-editable template in exon 3 of the CIITA locus comprises 80% identity to SEQ ID NO: 19. In some embodiments, the gRNA that directs a double-stranded break to insert a re-editable template in exon 3 of the CIITA locus comprises 85% identity to SEQ ID NO: 19. In some embodiments, the gRNA that directs a double-stranded break to insert a re-editable template in exon 3 of the CIITA locus comprises 90% identity to SEQ ID NO: 19. In some embodiments, the gRNA that directs a double-stranded break to insert a re-editable template in exon 3 of the CIITA locus comprises 95% identity to SEQ ID NO: 19. In some embodiments, the gRNA that directs a double-stranded break to insert a re-editable template in exon 3 of the CIITA locus comprises 99% identity to SEQ ID NO: 19. In some embodiments, the gRNA that directs a double-stranded break to insert a re-editable template in exon 3 of the CIITA locus comprises 100% identity to SEQ ID NO: 19.

In some embodiments, the gRNA in the cell that recognizes the re-editable template inserted in exon 3 of the CIITA locus comprises 70%, 80%, 90%, 95%, 99% or 100% identity to TGACTGATGTAAGACTAGTA (SEQ ID NO: 20). In some embodiments, the gRNA in the cell that recognizes the re-editable template inserted in exon 3 of the CIITA locus comprises 70% identity to SEQ ID NO: 20. In some embodiments, the gRNA in the cell that recognizes the re-editable template inserted in exon 3 of the CIITA locus comprises 75% identity to SEQ ID NO: 20. In some embodiments, the gRNA in the cell that recognizes the re-editable template inserted in exon 3 of the CIITA locus comprises 80% identity to SEQ ID NO: 20. In some embodiments, the gRNA in the cell that recognizes the re-editable template inserted in exon 3 of the CIITA locus comprises 85% identity to SEQ ID NO: 20. In some embodiments, the gRNA in the cell that recognizes the re-editable template inserted in exon 3 of the CIITA locus comprises 90% identity to SEQ ID NO: 20. In some embodiments, the gRNA in the cell that recognizes the re-editable template inserted in exon 3 of the CIITA locus comprises 95% identity to SEQ ID NO: 20. In some embodiments, the gRNA in the cell that recognizes the re-editable template inserted in exon 3 of the CIITA locus comprises 99% identity to SEQ ID NO: 18. In some embodiments, the gRNA in the cell that recognizes the re-editable template inserted in exon 3 of the CIITA locus comprises 100% identity to SEQ ID NO: 20.

In some embodiments, the re-editable template in the cell is defined by a sequence comprising 70%, 80%, 90%, 95%, 99% or 100% identity to AAATTTCCTTCTTCATCCAAGGGACTTTTCCTCCCAGAACCCGACACAGACACCA TCAACTGCGACCAGTTCAGCTGACTGATGTAAGACTAGTAAGGTGATGAAGAG ACCAGGGAGGCTTATGCCAATATCGGTGAGGAAGCACCTGAGCCCAGAAAAGGA CAATCAAGGGCAAGAGTTCTTTGCTGCCACTTGTCA (SEQ ID NO: 21). In some embodiments, the re-editable template in the cell is defined by a sequence comprising 70% identity to SEQ ID NO: 21. In some embodiments, the re-editable template in the cell is defined by a sequence comprising 75% identity to SEQ ID NO: 21. In some embodiments, the re-editable template in the cell is defined by a sequence comprising 80% identity to SEQ ID NO: 21. In some embodiments, the re-editable template in the cell is defined by a sequence comprising 85% identity to SEQ ID NO: 21. In some embodiments, the re-editable template in the cell is defined by a sequence comprising 90% identity to SEQ ID NO: 21. In some embodiments, the re-editable template in the cell is defined by a sequence comprising 95% identity to SEQ ID NO: 21. In some embodiments, the re-editable template in the cell is defined by a sequence comprising 99% identity to SEQ ID NO: 21. In some embodiments, the re-editable template in the cell is defined by a sequence comprising 100% identity to SEQ ID NO: 21.

In some embodiments, the re-editable template in the cell comprises an exogenous protein-coding gene.

In some embodiments, the exogenous protein-coding gene is an immune modulatory or cloaking gene. In some embodiments, the exogenous protein-coding gene is an immune modulatory gene. In some embodiments, the exogenous protein-coding gene is a cloaking gene.

In some embodiments, the exogenous gene in the cell expresses a protein that leads to cell death.

In some embodiments, the re-editable template of the cell comprises a chimeric antigen receptor (CAR) gene.

In some embodiments, provided herein is a method of modifying a gene locus in an engineered cell, comprising contacting the engineered cell provided herein with a genome editing system comprising a nucleic acid encoding a recombinant nuclease protein or a Cas protein and a guide RNA that specifically recognizes a PAM sequence comprised in the re-editable template, wherein the recombinant nuclease protein or Cas protein is capable of binding the guide RNA and editing the locus.

In some embodiments, the nucleic acid encoding a Cas protein is fused to an adenine or cytosine deaminase, and wherein the Cas protein fusion is capable of binding to the guide RNA and base editing the re-editable template.

In some aspects, provided herein is a re-editable template sequence, wherein the template sequence has no substantial sequence similarity to any region in the genome, and wherein the template sequence is recognizable by a genome editing system.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The FIGURE described below, which together make up the Drawings, are for illustration purposes only, not for limitation.

FIG. 1 is a schematic diagram showing a re-editable template being integrated into a WT locus by genome editing and homology-directed recombination, forming a re-editable target locus. A re-editable target locus comprises an integrated re-editable template and a homology arm on either side of the re-editable template.

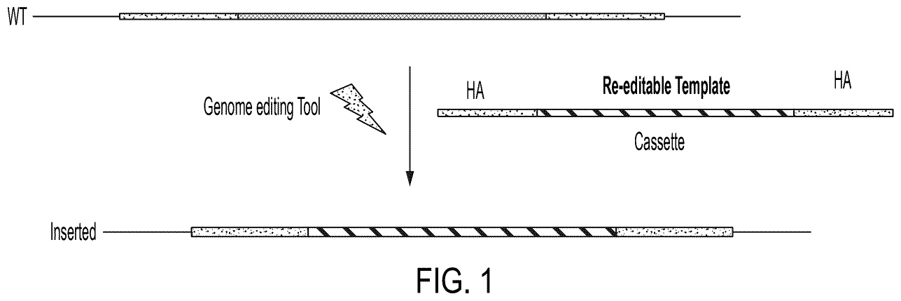

### DEFINITIONS

In order for the present invention to be more readily understood, certain terms are first defined below. Additional definitions for the following terms and other terms are set forth throughout the specification.

Approximately or about: As used herein, the term ‘approximately’ or ‘about’, as applied to one or more values of interest, refers to a value that is similar to a stated reference value. In certain embodiments, the term ‘approximately’ or ‘about’ refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Base editing: As used herein, ‘base editing’ is a CRISPR-Cas9-based genome editing technology that allows the introduction of point mutations in the DNA without generating DSBs. Two major classes of base editors include cytidine (or cytosine) base editors or CBEs allowing C>T conversions and adenine (or adenosine) base editors or ABEs allowing A>G conversions.

Biologically active: As used herein, the phrase ‘biologically active’ refers to a characteristic of any agent that has activity in a biological system, and particularly in an organism. For instance, an agent that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a ‘biologically active’ portion.

Homology Arm: As used herein, ‘homology arm’ refers to a flanking sequence, oneon either side of a re-editable template. A re-editable template is introduced to a wild-type target locus by homologous recombination between wild-type locus and donor template, thereby integrating the intervening re-editable template flanked by two homology arms. In some embodiments, each homology arm is between 50-500 nt. The sizes of the two homology arms may be the same or different relative to each other.

Homology directed repair (HDR): Homology directed repair (HDR) is one of the mechanisms by which DNA double-stranded breaks generated by CRISPR-endonuclease based genome editing is repaired. HDR requires a HDR donor template that contains sequences homologous to specific sequences flanking the cut site, defined as ‘homology arms’. HDR allows introduction of exogenous DNA template to obtain DNA editing within a genome. Briefly, HDR repair is carried out by the following steps: (a) The 5′ end of the DNA strand is resected, i.e. nucleotides are digested from the 5′ end at the cut site to generate a 3′ overhang; (b) The 3′ overhang acts as a substrate for a repair protein such as Rad51 protein that stimulates strand invasion by a donor repair template for base insertion or gene replacement; (c) The invasive strand displaces one strand of the homologous DNA duplex and pairs with the other, forming a hybrid DNA structure called displacement loop (D-loop); (d) The recombination intermediates are then resolved to repair DNA. Conservative HDR methods are precise and accurate and are carried out by classical double-stranded break repair, synthesis-dependent strand annealing or break-induced repair pathways.

Non-homologous end joining (NHEJ): Non-homologous end joining (NHEJ) is a mechanism by which DNA double-stranded breaks generated by CRISPR-endonuclease based genome editing is repaired. In NHEJ, the ends generated at a break site are directly ligated without need for a homologous template. NHEJ occurs in non-dividing and proliferating cells and is guided by short homologous DNA stretches or microhomologies, usually corresponding to the overhang. NHEJ is typically more error-prone than HDR. The general mechanism of NHEJ occurs as follows: (a) DNA end recognition and assembly and stabilization of the NHEJ complex comprising Ku70/Ku80 heterodimer at the DNA double strand break, which recruits other factors to the break site such as DNA-PKcs, XRCC4, DNA ligase IV, XRCC4-like factor (XLF), aprataxin and PNK like factor (APLF); (b) bridging of the DNA ends and promotion of end stability; (c) DNA end processing by enzymes such as Artemis, PNKP, APLF, Polymerases u and 2, Werner (WRN), aprataxin, and K; and (d) ligation of the broken ends by DNA ligase IV and dissolution of the NHEJ complex.

On-target Editing or On-target Editing Efficiency: On-target editing relates to specific and efficient gene editing at a desired target locus, while minimizing or preventing unintended or non-specific modifications to the genome at sites other than the target locus. ‘On-target editing’ or ‘On-target editing efficiency’ refers to the percent of cells edited at the on-target locus relative to total number of cells. Percent on-target editing is a measure of gene editing efficiency. For example, on-target editing of 50% means 50% of cells are edited at the on-target locus. In the case of CRISPR-Cas editing systems, on-target editing is determined by designing gRNAs which provide high on-target activity while having minimal off-target effects.

Off-target Editing: Off-target editing refers to non-specific and unintended genetic modifications that arise from genome editing methods. If repair complexes following cleavage of dsDNA bind at sequences other than the target sequence, due to homology or mismatch tolerance, the off-target sequences are cleaved causing non-specific genetic modifications including point mutations, deletions, insertions, inversions and/or translocations. Off-target binding results from a partial but sufficient match to the target sequence due to base mismatch tolerance in the linear sequence or bulge mismatch, i.e. off-target sites with a few missing bases or small deletions (RNA bulge) and off-target sites with extra bases or small insertions (DNA bulge) that are recognized by the gRNA. In some embodiments, the criteria specified to suppress off-target editing requires that coding genes in the genome have at least 3 mismatches relative to a template sequence that is recognizable by a specific guide RNA.

Prime Editing: As used herein, the term “Prime editing” refers to a versatile and precise genome editing method that directly writes new genetic information into a specified DNA site using a catalytically impaired Cas endonuclease fused to an engineered reverse transcriptase, programmed with a prime editing guide RNA (pegRNA) that both specifies the target site and encodes the desired edit. Prime editing is used to insert sequences about 30-40 nt in length. Prime editing edits sequences without generating a double-stranded break. It offers efficiency and product purity advantages over HDR, complementary strengths and weaknesses compared to base editing, and lower off-target editing than Cas nuclease at known Cas off-target sites.

RNA guide or Guide RNA (gRNA): refers to an RNA molecule that facilitates targeting of a Cas protein to a target locus. An gRNA is a guide RNA comprising a guide sequence (crRNA) and a Cas9 nuclease-recruiting sequence (tracrRNA). As used herein ‘sgRNA’ and ‘gRNA’ are used interchangeably.

Re-editable Template: As used herein, the term ‘re-editable template’ refers to a sequence that has no substantial sequence homology with any coding region of the genome, and is recognizable by a genome editing system.

Re-editable Locus or Re-editable Target or Re-editable Gene or Re-editable Site: As used herein, the terms ‘re-editable locus’, ‘re-editable target’, ‘re-editable gene’, ‘re-editable site’ are used interchangeably to refer to a locus, target, gene or site in the genome wherein a re-editable template flanked by two homology arms is integrated by homology driven recombination, and is recognizable by a genome editing system for subsequent modification.

Substantial Sequence Similarity: As used herein, the term ‘substantial sequence similarity’ is used interchangeably with ‘sequence homology’ and ‘sequence identity’ and refers to atleast 70%, 75%, 80%, 85%, 90% 95% or greater identity to a region in the genome. In some embodiments, substantial sequence similarity refers to 100% sequence identity.

### DETAILED DESCRIPTION

The present invention provides ‘Re-edit Technology,’ which is a flexible genome editing technology that is precise and specific for target loci, but also prepares edited loci for further manipulation by another precise genome editing system to achieve a variety of phenotypes or re-engineer cells of specific phenotypes within a short time for therapeutic use. The ‘Re-edit Technology’ of the present invention comprises methods of engineering re-editable target loci and cells with a re-editable template. The present invention provides, among other things, methods of engineering a re-editable target locus in a cell, comprising inserting an exogenous re-editable template sequence to a target locus and methods of engineering a re-editable cell comprising a re-editable template sequence flanked by two homology arms such that the re-editable template sequence is inserted into a target locus by homologous recombination, and methods of using the same. In some aspects, provided herein is a re-editable template sequence, wherein the template sequence has no substantial sequence similarity to any region in the genome, and wherein the template sequence is recognizable by a genome editing system. Various aspects of the invention are described in greater detail below.

### Engineering a Re-Editable Target Locus by Re-Edit Technology

The present invention provides, among other things, methods of engineering a re-editable target locus in a cell, comprising inserting an exogenous re-editable template sequence to a target locus, wherein the template sequence has no substantial sequence similarity to any coding region in the genome, and wherein the template sequence is recognizable by a genome editing system. In some embodiments, the template sequence is recognizable by integrases (e.g., phage-derived integrases, e.g., phiC31, Bxb1). As used herein, ‘exogenous’ refers to a sequence not typically expressed at a given locus.

### Re-Editable Template

A re-editable template sequence of the present invention is designed such that it has no substantial sequence similarity to any coding region in the genome. In some embodiments, the re-editable template has less than about 70%, 75%, 80%, 85%, 90%, 95% or 100% identity to any coding region in the genome. In some embodiments, the re-editable template has between about 70-75% identity to any coding region in the genome. In some embodiments, the re-editable template has between about 75-80% identity to any coding region in the genome. In some embodiments, the re-editable template has between about 80-85% identity to any coding region in the genome. In some embodiments, the re-editable template has between about 85-90% identity to any coding region in the genome. In some embodiments has between about 90-95% identity to any coding region in the genome. In some embodiments, the re-editable template has between about 95-100% identity to any coding region in the genome. In some embodiments, the re-editable template has less than 70% identity to any coding region in the genome. In some embodiments, the re-editable template has less than 75% identity to any coding region in the genome. In some embodiments, the re-editable template has less than 80% identity to any coding region in the genome. In some embodiments, the re-editable template has less than 85% identity to any coding region in the genome. In some embodiments, the re-editable template has less than 90% identity to any coding region in the genome. In some embodiments, the re-editable template has less than 95% identity to any coding region in the genome. In some embodiments, the re-editable template has less than 100% identity to any coding region in the genome.

Accordingly, off-target editing is minimized or eliminated by the re-editable template of the present invention. Off-target editing is a major limitation of genome editing technologies that needs to be suppressed to provide for safe and widespread use of gene therapy in a variety of therapeutic applications. Briefly, off-target editing causes double-stranded DNA breaks at sequences different from the intended target site, leading to unwanted mutations and gene disruptions, leading to toxicity or lethality. However, off-target editing can also occur at the target site, for example, a single base pair substitution in a protospacer sequence at the target site may cause re-cutting by a Cas enzyme at the same target site, removing the desired genome modification by template repair pathways. Often, additional target-site disrupting mutations are introduced in the repair template to prevent re-cutting, but these may affect regulatory motifs or result in non-synonymous mutations.

Off-target cleavage in CRISPR-Cas systems results from recognition of complementary genomic sites by gRNAs, and some approaches to minimize this includes Cas9 variants with lower off-target specificity, e.g. HF-Cas9, eCas9, and HypaCas9. In some embodiments of the present invention, the Cas9 is a variant Cas9 or a Cas9 homolog.

The present invention provides a re-editable template that prevents undesirable re-cutting by the same genome editing tool as used to insert the re-editable template, but instead prepares the template for subsequent specific modification by another genome editing tool. In some embodiments, the genome editing tool is a TALEN, Zinc finger nuclease, meganuclease, CRISPR-Cas or related nuclease, guided by a nucleic acid, e.g. RNA or a protein. In some embodiments, the genome editing tool is a sequence-specific recombinase or integrase, e.g. phiC31 with its cognate attP or attB recognition sequence. In some embodiments, the target is a pseudo attP or attB site in the genome recognizable by a recombinase or integrase. The re-editable template of the present invention allows subsequent targeted modification of cells by a different genome editing nuclease than the one used to insert the re-editable template, thereby facilitating rapid development of multiple different types of mutations in platform cells for treating multiple different indications.

The re-editable template sequence designed determines the suitability of the genome editing system for subsequent editing. For example, in some embodiments, wherein a sequence is inserted 5′ to an existing PAM site, a new cleavage site is created within proximity of the previously existing PAM site, making the template sequence re-editable as the PAM site is specified in the homology arm. In some embodiments, if a re-editable template has no 5′-NGG-3′ PAM, it would not be possible to subsequently modify the re-editable locus by a Cas9 nuclease, but it would be recognizable and editable by TALEN, for example, or another Cas protein. Thus in various embodiments, a re-editable template is designed to prevent re-cutting and off-target effects, while allowing subsequent precise genome modification.

In some embodiments, any coding region in the genome contains at least 3 mismatches relative to the template sequence recognizable by a guide RNA. In some embodiments, any coding region in the genome contains at least 1 mismatch relative to the template sequence recognizable by a guide RNA. In some embodiments, any coding region in the genome contains at least 2 mismatches relative to the template sequence recognizable by a guide RNA. In some embodiments, any coding region in the genome contains at least 4 mismatches relative to the template sequence recognizable by a guide RNA. In some embodiments, any coding region in the genome contains at least 5 mismatches relative to the template sequence recognizable by a guide RNA. In some embodiments, any coding region in the genome contains at least 6 mismatches relative to the template sequence recognizable by a guide RNA. In some embodiments, any coding region in the genome contains at least 7 mismatches relative to the template sequence recognizable by a guide RNA. In some embodiments, any coding region in the genome contains at least 8 mismatches relative to the template sequence recognizable by a guide RNA. In some embodiments, any coding region in the genome contains at least 9 mismatches relative to the template sequence recognizable by a guide RNA. In some embodiments, any coding region in the genome contains less than 10 mismatches relative to the template sequence recognizable by a guide RNA. It is evident that a template sequence is recognizable by a guide RNA, when a nuclease with mismatch tolerance is subsequently recruited and cleavage occurs.

A re-editable template of the present invention is less than about 500 nt. In some embodiments, the re-editable template is between about 10-500 nt. In some embodiments, the re-editable template is between about 10-100 nt some embodiments, the re-editable template is between about 10-20 nt. In some embodiments, the re-editable template is between about 20-30 nt. In some embodiments, the re-editable template is between about 30-40 nt. In some embodiments, the re-editable template is between about 40-50 nt. In some embodiments, the re-editable template is between about 50-60 nt. In some embodiments, the re-editable template is between about 60-70 nt. In some embodiments, the re-editable template is between about 70-80 nt. In some embodiments, the re-editable template is between about 80-90 nt. In some embodiments, the re-editable template is between about 90-100 nt. In some embodiments, the re-editable template is between about 100-200 nt. In some embodiments, the re-editable template is between about 200-300 nt. In some embodiments, the re-editable template is between about 300-400 nt. In some embodiments, the re-editable template is between about 400-500 nt.

In some embodiments, an entire exon is replaced with a re-editable template. In some embodiments, one or more exons are replaced with a re-editable template, for example, with different PAMs. In some embodiments, a re-editable template is inserted within a non-coding region.

In some embodiments, wherein the genome editing system is a CRISPR-Cas system, the Cas protein is a Type II Cas enzyme. In some embodiments, the Cas protein is Cas9, Cas12a or Cas12b. Type II nucleases include Cas9, a dual-RNA-guided nuclease which requires both CRISPR RNA (crRNA) and tracrRNA and contains both HNH and RuvC nuclease domains; Cas12a, a single-RNA-guided nuclease which only requires crRNA and contains a single RuvC domain; and Cas12b, a dual RNA-guided nuclease containing a single RuvC domain and requiring both crRNA and tracrRNA.

In some embodiments, the Cas protein is an enzymatically dead Cas protein or nickase. In some embodiments, the Cas protein is Cas9 D10A or Cas9 H840A. In some embodiments, the Cas nuclease, TALEN or ZFN is fused to a FokI nuclease or related nuclease domain. Dead Cas9 is fused with FokI nuclease (fCas9) to improve DNA cleavage specificity. DNA cleavage by fCas9 requires association of two fCas9 monomers that simultaneously bind target sites 15 or 25 base pairs apart. In human cells, fCas9 modified target DNA sites with greater than 140-fold higher efficacy than wild-type Cas9. (Guilinger, Thompson and Liu, 2014, Fusion of catalytically inactive Cas9 to FokI nuclease improves the specificity of genome modification, Nature Biotechnology, 32, p. 577-582). In some embodiments, dead Cas9 is fused to a M-MLV reverse transcriptase. In some embodiments, Cas9 H840A is fused to a M-MLV reverse transcriptase for the prime editing.

In various embodiments of the present invention, wherein the genome editing system is a CRISPR-Cas system, a re-editable template comprises a Protospacer Adjacent Motif (PAM). A PAM is typically a DNA sequence between 2-6 nt in length adjacent to a CRISPR locus that is targeted for cleavage by a Cas-related nuclease in a CRISPR-Cas genome editing system. A Cas enzyme is directed to bind to a DNA sequence adjacent to a PAM sequence and thus carry out DNA cleavage at precise sites. In some embodiments, the re-editable template comprises a 5′-NGG-3′ PAM sequence, where N is any nucleotide followed by two guanine nucleotides, which is recognized by Cas9 enzyme from Streptococcus pyogenes (SpCas9). In some embodiments, the re-editable template comprises a 5′-NGG-3′ PAM and the nuclease is Cas9. Further, S. pyogenes Cas9 mutants that have altered specificity or recognize novel PAM sequences have been identified. SaCas9 (from Staphylococcus aureus) specifically recognizes the short PAM sequence 5′-NGRR (N)-3′. Other Cas nucleases, including Cas9 from other species, recognize longer PAMs or have higher specificity to minimize off-target cutting. An appropriate Cas nuclease and corresponding PAM is thus selected for the re-editable template design. The re-editable template of the present invention comprises in various embodiments, different PAMs associated with the Cas9 from different species. For example, Neisseria meningitides (NmeCas9) specifically recognizes NNNNGATT, Campylobacter jejuni (CjCas9) specifically recognizes NNNNRYAC, Streptococcus thermophilus (StCas9) recognizes NNAGAAW, Lachnospiraceae bacterium (LbCpf1, Cas12a) recognizes TTTV, Acidaminococcus sp. (AsCpf1, Cas12a) recognizes TTTV, Alicyclobacillus acidiphilus (AacCas12b) recognizes TTN, Bacillus hisashii (BhCas12bv4) recognizes ATTN, TTTN, GTTN, and uncultivated archea (Cas14) recognizes T-rich PAM sequences, e.g., TTTA.

Some PAM sequences are engineered to expand the repertoire of Cas9 targets, for example, Francisella novicida Cas9 recognizes the canonical 5′-NGG-3′ PAM but is engineered to recognize 5′-YG-3′, where Y is any pyrimidine. In addition to CRISPR-Cas9, Francisella novicida CRISPR-Cpf1 recognizes PAM 5′-TTTN-3′ or 5′-YTN-3′. CRISPR-Cas13a (C2c2) is an RNA-guided CRISPR system that does not rely on a PAM recognition sequence, but instead requires a Protospacer Flanking Site (PFS), which is typically a nucleotide other than guanine, which is known to reduce efficacy.

In some embodiments, a re-editable template of the present invention thus has no substantial sequence similarity with any coding region of the genome, has specificity for recognition by a desired genome editing system that is not the same as the one used to insert it into a target locus, and is of various lengths, for example, 10-500 nt, comprising exogenous sequences of interest that are not usually expressed at that locus. In some embodiments, the exogenous sequences are non-coding sequences.

In some embodiments, a re-editable template is defined by a sequence comprising 70%, 80%, 90%, 95%, 99% or 100% identity to

(SEQ ID NO: 5)
ATTCCTGAAGCTGACAGCATTCGGGCCGAGATGTCCAGGTCCTAATGAT
TAGCTGTGCTCGCCCTGCTCTCTCTGTCTGGCCTGGAGGCTATTCAGCG
TGAGTCTCTCCTACCCTCCCGCTCTGGTCCTTCCTCTCCCGCTCTGCAC
CCTCTGTGGCCCT,
or
(SEQ ID NO: 6)
AAAACGGGAAAGTCCCTCTCTCTAACCTGGCACTGCGTCGCTGGCTTGG
AGACAGGTGACGGTCCCTGCGGGCCTTGTCCTGATTGGCTGGGCACGCG
TTTAATATAAGTGGAGGCGTCGCGCTGGCGGGCATTCCTGAAGCTGACA
GCATTCGGGCCGAGATGTCGTAGAGCGTGTGACTAGCTGTACTGGAGCT
GTGAAGCTAATCCGGTCTGGAAGCCATTCAGCGTGAGTCTCTCCTACCC
TCCCGCTCTGGTCCTTCCTCTCCCGCTCTGCACCCTCTGTGGCCCTCGC
TGTGCTCTCTCGCTCCGTGACTTCCCTTCTCCAAGTTCTCCTTGGT

In some embodiments, a re-editable template is defined by a sequence comprising 70%, 80%, 90%, 95%, 99% or 100% identity to

(SEQ ID NO: 12)
AAATGTAAACACTTGGTGCCTGATATAGCTTGACACCAAGTTAGCCCCA
AGTGAAATACCCTGGCAATATTAATGTGTCTTTTCCCGATATTCCTCAG
GTACCCCCTAGATCCAATAGTAGAGTAGGTGACCAGCCTAGAACGGAGC
CTGTAGGGTGAACCACGTGACCCTGTAACAGTGGGGTAAGTCTTACATT
CTTTTGTAAGCTGCTGAAAGTTGTGTATGAGTAGTCATATCATAAAGCT
GCTTTGATATAAAAAAGGTCTATGGCCATACTACCC.

In some embodiments, the re-editable template is defined by a sequence comprising 70%, 80%, 90%, 95%, 99% or 100% identity to

(SEQ ID NO: 18)
TTCTGCCTCTTTCCAACACCCTGTGAGGTGACTGAGCATTGTCTTCCCT
CCCAGGCAGTTCCCAGTGTGCTACCATGGAGTTGTGACCCCTATAATGA
GACCTGGCTGGAGAAGAAGAGATTGAGCTCTACTCAGGTGGGCCCTCCT
CC.

In some embodiments, the re-editable template is defined by a sequence comprising 70%, 80%, 90%, 95%, 99% or 100% identity to

(SEQ ID NO: 21)
AAATTTCCTTCTTCATCCAAGGGACTTTTCCTCCCAGAACCCGACACAG
ACACCATCAACTGCGACCAGTTCAGCTGACTGATGTAAGACTAGTAAGG
TGATGAAGAGACCAGGGAGGCTTATGCCAATATCGGTGAGGAAGCACCT
GAGCCCAGAAAAGGACAATCAAGGGCAAGAGTTCTTTGCTGCCACTTGT
CA.

### Generating a Re-Editable Target Locus by Introducing a Re-Editable Template

The present invention provides a method of engineering a re-editable target locus in a cell, comprising inserting an exogenous re-editable template sequence to a target locus, wherein the template sequence has no substantial sequence similarity to any region in the genome, and wherein the template sequence is recognizable by a genome editing system.

Exogenous re-editable template refers to a sequence other than the sequence endogenous to that locus, i.e., the sequence typically present in the genome at the target locus. The exogenous re-editable template, includes in some embodiments, wild-type sequence expressed at another locus, or in the genome of another species, or another cell of the same or different species. The exogenous re-editable template, also includes, in some embodiments, modified or engineered recombinant sequences. The exogenous re-editable template, includes in some embodiments, a gene that encodes a protein.

In some embodiments, the re-editable template sequence is flanked by two homology arms such that the re-editable template sequence is inserted into the target locus by homologous recombination.

Homologous recombination is a type of genetic recombination in which nucleotide sequences are exchanged between two similar or identical molecules of DNA, i.e., flanking homology arms. Briefly, homologous recombination is initiated by a double-stranded break, which is repaired by a cell. Genomic editing, for examples, induces a DNA break at one or more sites, such as a double-stranded break (DSB) at a target locus in the genome (or target genomic region). DNA cleavage causes cellular enzymes to repair the break through either an error prone Non-homologous end-joining pathway (NHEJ) or an error-free Homology directed repair (HDR) pathway.

In NHEJ, the DNA lesion is repaired by fusing the two ends of the DNA break in a series of enzymatic processes involving Ku70/80 heterodimer and DNA dependent protein kinase (DNA-PKc) enzymes. The repair mechanism involves tethering and alignment of two DNA ends, resection, elongation and ligation resulting in the formation of small insertion or deletion mutations (indels) at the break site. Indels introduced into the coding sequence of a gene can cause either premature stop codon or frame-shift mutations that lead to the production of nonfunctional, truncated proteins. Using NHEJ to generate indels that knock out gene expression can create two unique alleles in a gene.

Homology-directed repair (HDR) is another mechanism by which cells repair double-stranded DNA breaks. HDR requires a HDR donor template that contains sequences homologous to the specific sequence flanking the cut site, defined as ‘homology arms’. The HDR pathway involves a repair protein such as Rad51 that stimulates strand invasion by a donor repair template for base insertion or gene replacement. HDR allows introduction of exogenous DNA template to obtain DNA editing within a genome.

The optimal length of homology arms has been reported to be as little as 30 nt in length on either side of the DSB, and it has been demonstrated that asymmetric donor oligos can improve HDR. HDR efficiency is highest when the intended edit is placed near the DSB and is greatly reduced at loci distal to this event. In addition, reports have indicated that there may be a preference for utilizing a donor oligo with sequences either complementary or non-complementary to the gRNA.

Since re-cutting of dsDNA by CRISPR-Cas lowers HDR efficiency, blocking mutations are incorporated. Thus HDR using a homozygous frame shift mutation (e.g. an indel resulting in a premature stop codon) with silent blocking mutation(s) generates two identical alleles. HDR occurs at very low frequency during S and G2 phases of DNA replication. The most common form of HDR is homologous recombination. The HDR mechanism can only be used by the cell when there is a homologous piece of DNA present in the nucleus, mostly in G2 and S phase of the cell cycle. Other examples of homology-directed repair include single-strand annealing and breakage-induced replication.

The intervening re-editable template sequence herein is integrated into the genome by homologous recombination, generating a re-editable target locus comprising the re-editable template and flanking homology arms. The sizes of the two homology arms may be the same or different relative to each other.

In some embodiments, homology arm is between about 50 to 500 nt. In some embodiments, homology arm is 30 nt. In some embodiments, homology arms are between about 50 nt. In some embodiments, homology arm is about 100 nt in length. In some embodiments, homology arm is about 150 nt in length. In some embodiments, homology arm is about 200 nt in length. In some embodiments, homology arm is about 250 nt in length. In some embodiments, homology arm is about 300 nt in length. In some embodiments, homology arm is about 350 nt in length. In some embodiments, homology arm is about 400 nt in length. In some embodiments, homology arm is about 450 nt in length. In some embodiments, homology arm is about 500 nt in length.

In some embodiments, the subsequent modification uses a gRNA guided Cas nuclease to generate a double-stranded break and integrate an exogenous protein-coding gene to a target locus.

### Target Locus

A target locus of the present invention is any genomic locus wherein a re-editable template is inserted to allow subsequent modification of the genome at the desired locus. For example, a target gene locus or target locus is a locus where a gene function is knocked out and subsequent genome editing is carried out to integrate an exogenous gene. In some embodiments, target locus is a locus where a gene function is not disrupted or knocked out.

In some embodiments, the target locus is at least one locus selected from the group consisting of a ubiquitously expressed gene, cell division related gene, and gene with expression restricted to specific cell types. In some embodiments, the target locus is a ubiquitously expressed gene, e.g. HLA Class I genes, e.g. B2M gene. HLA class I molecules are expressed on the surface of almost all nucleated cells. In some embodiments, the target locus is a cell division related gene, e.g. a cell cycle checkpoint gene. In some embodiments, the target locus is a gene with expression restricted to specific cell types, e.g. HLA Class II gene. HLA Class II molecules are expressed only on B lymphocytes, antigen-presenting cells (monocytes, macrophages, and dendritic cells), and activated T lymphocytes.

Genetic loci involved in the rejection of foreign cells in transplantation are known as the major histocompatibility complex (MHC). The MHC encode highly polymorphic cell surface molecules. Human MHC is also known as the HLA (Human Leukocyte Antigen) system because these antigens were first identified and characterized using alloantibodies against leukocytes. Molecular HLA allele typing is typically performed for matching HLA class I and class II alleles in allogeneic stem cell transplantation to avoid transplant rejection. HLA molecules play a role in immune regulation and detection of self and non-self antigens. In some embodiments, the target locus is a Class I or Class II HLA gene. In some embodiments, the target locus is B2M. In some embodiments, a target locus is CIITA.

In some embodiments, a target locus is a safe harbor locus, which is a location in the genome that allows for expression of an exogenous gene without risk of affecting neighboring endogenous genes, e.g. ROSA26, AAVS1, CLYBL, H11, COL1A1, CCR5, collagen, HTRP, GAPDH, ACTB, ACTG1, TCR, RUNX1, POLR2a. In some embodiments, a target locus is a locus desirable for gene interruption, e.g. TAP1, TAP2 or tapasin, beta-2 microglobulin (B2M), NLRC5, PD1, LAG3, TIM3, RFXANK, CIITA, RFX5, RFXAP, among others.

In some embodiments, the target locus is an attachment site in the genome for a recombinase or integrase, for example, an attP or attB site in the genome. In some embodiments, the integrase is a phage-derived integrase. In some embodiments, the target locus is a pseudo attP or attB site in the genome. In some embodiments, the target is a minimal attP or attB sequence.

In some embodiments, the integrase is a BxB1. In some embodiments, the BxB1 attP site is GTCGTGGTTTGTCTGGTCAACCACCgcggtctcAGTGGTGTACGGTACAAACCCCGA (SEQ ID NO: 26). In some embodiments, the BxB1 attB site is TCGGCCGGCTTGTCGACGACGgeggtctcCGTCGTCAGGATCATCCGGGC (SEQ ID NO: 27). In some embodiments, the integrase is phiC31. In some embodiments, the phiC31 attP site is GTGCCCCAACTGGGGTAACCTttGAGTTCTCTCAGTTGGGGG (SEQ ID NO: 28). In some embodiments, the phiC31 attB site is TGCGGGTGCCAGGGCGTGCCCttGGGCTCCCCGGGCGCGTACTCC (SEQ ID NO: 29). In bold and lower case are the core nucleotides that are critical for the synapse formation during recombination between the attP and attB sequences (via cognate integrase), while the flanking sequence facilitates recognition/binding by the integrase.

### Exogenous Gene

An exogenous gene is one that is not typically present at that locus, or expressed from that locus. In some embodiments, the exogenous gene is a protein coding gene. In some embodiments, the exogenous gene is non-coding. Exogenous gene includes any gene expressing a protein that may otherwise be expressed at other loci or other cells, and includes wild-type sequences. For example, the exogenous gene includes PD-L1, FASL, CD47, CD24, B2M, HLA-E, HLA-G, CD200, CCL21, MFGE8, H2-M3, SPI6, among others.

In some embodiments, the exogenous gene encodes a safety switch and expresses a protein that leads to cell death, wherein the gene is selected from the group consisting of herpes simplex virus thymidine kinase (HSV-tk), iCaspase8, iCaspase9, iCaspase3, iCaspase7, cytosine deaminase, modified EGFR, B-cell CD20, among others. In some embodiments, the safety switch is linked to endogenous gene expression (e.g. via cleavable 2A linker or IRES). In some embodiments, the safety switch is linked to endogenous gene expression via cleavable 2A linker. In some embodiments, the safety switch is linked to endogenous gene expression via internal ribosome entry site (IRES) element. In some embodiments, the safety switch is driven by a synthetic promoter (e.g. EFIA, CAG). In some embodiments, the safety switch is driven by EFIA promoter. In some embodiments, the safety switch is driven by CAG promoter.

Stimulating suicide genes is important in killing cancer cells, unwanted cells, or other proliferative cells in tissues and organs, and to make cells more sensitive to chemotherapy. Most suicide genes mediate this by coding for viral or bacterial enzymes that convert an inactive drug into toxic antimetabolities that inhibit nucleic acid synthesis. In some embodiments, the exogenous gene expresses a suicide gene that is activated by an inducing molecule and triggers cell death of the hypoimmune iPSCs or cells derived from it. For example, herpes simplex virus (HSV)-tk is triggered by ganciclovir. E. coli cytosine deaminase gene (EC-CD) is triggered by 5-fluorocytosine (5-FC). Suicide genes must be introduced in very specific cells and at targeted loci, so that they do not have off-target effects. In some embodiments of this invention, providing suicide genes in a re-editable template provides a means to control expression at desired times and at precise loci in the cell.

In some embodiments, the protein coding gene is a cloaking gene that protects cells from recognition by NK cells, generating hypoimmune cells (e.g., iPSCs (e.g., undifferentiated iPSCs and in some embodiments, including progenitor cells and/or differentiated cells), stem cells, progenitor cells, immune cells including T cells and NK cells, or any other differentiated cells). In some embodiments, the protein-coding gene triggers cell termination in the presence of an inducing molecule. In some embodiments, the protein coding gene expresses one or more proteins that increase efficacy of a chimeric antigen receptor (CAR) product.

Exogenous genes may comprise a gene encoding transcription factors, receptors, signaling molecules, pharmaceutically active peptides, or a gene encoding a protein promoting a variety of cellular functions, including but not limited to trafficking, homing, engraftment, self-renewal, persistence, viability, and/or survival of the iPSCs or iPSC derived cells used in tissue engineering.

In some embodiments, the exogenous gene encodes an RNA product, including but not limited to siRNA, shRNA, microRNA, antisense nucleic acids, circRNAs and lncRNAs. In some embodiments, the re-editable template includes one or more constitutive promoters, inducible promoters, temporal-specific promoters, and/or tissue or cell type specific promoters to drive expression of the exogenous gene.

### Genome Editing Systems

Double-stranded breaks greatly increase the efficiency of homology dependent repair in mammalian cells, and targeted nucleases guided by RNA or protein are used in genome editing systems to create modifications at precise target sites. Genome editing systems include protein guided systems such as Zinc Finger Nucleases or TALENs, and RNA guided systems such as

### Zinc Finger Nucleases (ZENs)

In some embodiments of the present invention, the re-editable template is designed to be recognizable by a Zinc finger nuclease (ZFN) system. ZFNs comprise a site-specific DNA binding domain and a non-sequence specific cleavage domain. Zinc finger domains comprise a modular array of Cys2His₂ zinc fingers, each of which is about 30 amino acids and recognizes three base pairs of DNA. Target recognition is determined by the amino acid sequence of each zinc finger, the number of zinc fingers in the array and the interaction with a FokI endonuclease domain. Two ZFN molecules bind to the target site in an appropriate orientation, requiring recognition of 6 sites for DNA cleavage by zinc finger nuclease comprising a fusion of a FokI and zinc fingers, creating double-stranded breaks at specific loci resulting in subsequent endogenous NHEJ or HDR repair systems. Zinc finger nucleases are optimized by engineering DNA binding and catalytic domains. In some embodiments, ZFNs containing 3-6 fingers can interact with a 9-18 nt of DNA, thus precise target recognition is specified by 18-36 nt of DNA at each cleavage site. Further, linkers spanning finger-finger and finger-FokI cleavage domain junctions vastly increase the number of ZFN configurations for targeting precise genome editing.

### Transcription Activator-Like Effector Nucleases (TALENs)

In some embodiments of the present invention, the re-editable template is designed to be recognizable by a TALEN system. TALENs are fusion proteins of bacterial TALE protein derived from Xanthomonas and FokI endonuclease. A TALE motif contains 33-35 amino acid repeat domains that each recognizes one nucleotide in DNA. An array of TALEs can bind a stretch of DNA e.g. 30-40 nt, with the specificity that a TALE binding site starts with a T base. The TALE protein has two hypervariable amino acids known as Repeat Variable Diresidues that provide specificity. FokI endonuclease requires dimerization and binding of TALENs at opposite strands to the target DNA for cleavage.

### Clustered Regularly Interspaced Short Palindromic Repeats (CRISPR)-Cas Endonucleases

In some embodiments of the present invention, the re-editable template is designed to be recognizable by a CRISPR-Cas system. A CRISPR genome editing system is an RNA guided editing system that comprises a Cas nuclease, a trans-activating crRNA (tracrRNA) and a CRISPR RNA (crRNA) that recognizes a target sequence. The target sequence must be followed by a Protospacer Adjacent Motif (PAM). Typically, CRISPR-Cas systems recognize between 20-25 nt, e.g. 22 nt and a PAM sequence. Each Cas9 protein has a specific PAM sequence, e.g., Cas9 recognizes 5′-NGG-3′ and generates a double-stranded break. Mutant Cas9 variants called nickases generate a single-strand break. Since target recognition in the CRISPR-Cas system is guided by RNA, such gRNAs can be designed for any genomic target, with criteria that minimize off-target editing. Further, multiple sites are able to be modified simultaneously in a multiplex manner, if desired.

Cas9 variants and homologs, such as CRISPR-Cas12 (Cpf1) and CRISPR-Cas13a (C2c2), recognize different PAMs. In some embodiments, a Cas protein is Cas9, Cas12a or Cas12b. In some embodiments, the Cas protein is an enzymatically dead Cas protein or nickase. In some embodiments, the nickase is Cas9 D10A. Further, in some embodiments, a Cas9 protein is fused to a FokI endonuclease.

### Design of Guide RNA

RNA guide and Guide RNA are used interchangeably herein. Appropriate guide RNA sequences are carefully designed based on the target locus location and sequence. A guide RNA must be highly active and have high efficiency, while also minimizing off-target editing. In some embodiments, a guide RNA comprises about 20 nt followed by a specific PAM sequence, for example, 20 nt followed by 5′-NGG-3′ for SpCas9. For homology dependent repair, a gRNA sequence recognizes DNA sequence within about 30 nt of the desired site of editing.

In some embodiments, an appropriate guide RNA is selected to include 20 nt followed by a PAM sequence comprising greater than 2 mismatches. In some embodiments, the guide RNA is selected to include 20 nt followed by a PAM sequence comprising 3 mismatches. For example, in some embodiments, the guide RNA is selected using tools known in the art for guide RNA selection using the criteria of greater than 50% for on-target efficiency and no protein coding gene with less than 3 mismatches in the predicted off-target list. No target site with 70% or greater homology within the human genome is included, i.e. the gRNA targets a site with less than 70% homology to another region in the genome, thereby reducing off-target cleavage. No target site with 70%, 75%, 80%, 85%, 90%, 95% or greater homology within the human genome is included. Various gRNA design tools are known in the art, including but not limited to, E-CRISP, CHOP-CHOP, CRISPR-ERA, Broad gRNA, Addgene, IDT sequence checker, Benchling and Desktop Genomics, which, in various embodiments, are used for guide RNA design. In some embodiments, guide RNA design targets within 50 nt to 500 nt of the transcription start site for CRISPR activation technology, near the transcription start site for CRISPR interference, within specific exon, intron sequences for knocking in genes, and within protein-coding exons for knocking out sequences.

In some embodiments, a gRNA that directs a double-stranded break to insert a re-editable template in a B2M exon 1 locus comprises 70%, 80%, 90%, 95%, 99% or 100% identity to GAGTAGCGCGAGCACAGCTA (SEQ ID NO: 1), AGGGTAGGAGAGACTCACGC (SEQ ID NO: 2) or GGCCGAGATGTCTCGCTCCG (SEQ ID NO: 3).

In some embodiments, the gRNA that recognizes the re-editable template inserted in a B2M exon 1 locus comprises 70%, 80%, 90%, 95%, 99% or 100% identity to GAGCGTGTGACTAGCTGTAC (SEQ ID NO: 4). In some embodiments, the gRNA that recognizes the re-editable template inserted in a B2M exon 1 locus comprises 70% identity to GAGCGTGTGACTAGCTGTAC (SEQ ID NO: 4). In some embodiments, the gRNA that recognizes the re-editable template inserted in a B2M exon 1 locus comprises 80% identity to GAGCGTGTGACTAGCTGTAC (SEQ ID NO: 4). In some embodiments, the gRNA that recognizes the re-editable template inserted in a B2M exon 1 locus comprises 90% identity to GAGCGTGTGACTAGCTGTAC (SEQ ID NO: 4). In some embodiments, the gRNA that recognizes the re-editable template inserted in a B2M exon 1 locus comprises 95% identity to GAGCGTGTGACTAGCTGTAC (SEQ ID NO: 4). In some embodiments, the gRNA that recognizes the re-editable template inserted in a B2M exon 1 locus comprises 99% identity to GAGCGTGTGACTAGCTGTAC (SEQ ID NO: 4). In some embodiments, the gRNA that recognizes the re-editable template inserted in a B2M exon 1 locus comprises 100% identity to GAGCGTGTGACTAGCTGTAC (SEQ ID NO: 4).

In some embodiments, a gRNA that directs a double-stranded break to insert a re-editable template in a B2M exon 2 locus comprises 70%, 80%, 90%, 95%, 99% or 100% identity to AAGTCAACTTCAATGTCGGA (SEQ ID NO: 7), AGTCACATGGTTCACACGGC (SEQ ID NO: 8), or ACTTGTCTTTCAGCAAGGAC (SEQ ID NO: 9).

In some embodiments, a gRNA that recognizes the re-editable template inserted in a B2M exon 2 locus comprises 70%, 80%, 90%, 95%, 99% or 100% identity to CCTAGATCCAATAGTAGAGT (SEQ ID NO: 10) or GGTCACGTGGTTCACCCTAC (SEQ ID NO: 11).

In some embodiments, a gRNA that directs a double-stranded break to insert a re-editable template in exon 2 of the CIITA locus comprises 70%, 80%, 90%, 95%, 99% or 100% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15). In some embodiments, a gRNA that directs a double-stranded break to insert a re-editable template in exon 2 of the CIITA locus comprises 70% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15). In some embodiments, a gRNA that directs a double-stranded break to insert a re-editable template in exon 2 of the CIITA locus comprises 80% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15). In some embodiments, a gRNA that directs a double-stranded break to insert a re-editable template in exon 2 of the CIITA locus comprises 90% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15). In some embodiments, a gRNA that directs a double-stranded break to insert a re-editable template in exon 2 of the CIITA locus comprises 95% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15). In some embodiments, a gRNA that directs a double-stranded break to insert a re-editable template in exon 2 of the CIITA locus comprises 99% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15). In some embodiments, a gRNA that directs a double-stranded break to insert a re-editable template in exon 2 of the CIITA locus comprises 100% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15).

In some embodiments, the gRNA that recognizes the re-editable template inserted in a CIITA exon 2 locus comprises 70%, 80%, 90%, 95%, 99% or 100% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 70% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 80% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 90% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 95% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 99% identity to GTGACCCCTATAATGAGACC (SEQ ID NO: 16) or CAGTTCCCAGTGTGCTACCA (SEQ ID NO: 17). In some embodiments, the gRNA that recognizes the re-editable template inserted in a CIITA exon 2 locus comprises 100% identity to GTGACCCCTATAATGAGACC (SEQ ID NO: 16) or CAGTTCCCAGTGTGCTACCA (SEQ ID NO: 17).

In some embodiments, a gRNA that directs a double-stranded break to insert a re-editable template in exon 3 of the CIITA locus comprises 70%, 80%, 90%, 95%, 99% or 100% identity to AGGCTGTTGTGTGACATGGA (SEQ ID NO: 19). In some embodiments, a gRNA that directs a double-stranded break to insert a re-editable template in exon 3 of the CIITA locus comprises 70% identity to AGGCTGTTGTGTGACATGGA (SEQ ID NO: 19). In some embodiments, a gRNA that directs a double-stranded break to insert a re-editable template in exon 3 of the CIITA locus comprises 80% identity to AGGCTGTTGTGTGACATGGA (SEQ ID NO: 19). In some embodiments, a gRNA that directs a double-stranded break to insert a re-editable template in exon 3 of the CIITA locus comprises 90% identity to AGGCTGTTGTGTGACATGGA (SEQ ID NO: 19). In some embodiments, a gRNA that directs a double-stranded break to insert a re-editable template in exon 3 of the CIITA locus comprises 95% identity to AGGCTGTTGTGTGACATGGA (SEQ ID NO: 19). In some embodiments, a gRNA that directs a double-stranded break to insert a re-editable template in exon 3 of the CIITA locus comprises 99% identity to AGGCTGTTGTGTGACATGGA (SEQ ID NO: 19). In some embodiments, a gRNA that directs a double-stranded break to insert a re-editable template in exon 3 of the CIITA locus comprises 100% identity to AGGCTGTTGTGTGACATGGA (SEQ ID NO: 19).

In some embodiments, the gRNA that recognizes the re-editable template inserted in exon 3 of the CIITA locus comprises 70%, 80%, 90%, 95%, 99% or 100% identity to TGACTGATGTAAGACTAGTA (SEQ ID NO: 20). In some embodiments, the gRNA that recognizes the re-editable template inserted in exon 3 of the CIITA locus comprises 70% identity to TGACTGATGTAAGACTAGTA (SEQ ID NO: 20). In some embodiments, the gRNA that recognizes the re-editable template inserted in exon 3 of the CIITA locus comprises 80% identity to TGACTGATGTAAGACTAGTA (SEQ ID NO: 20). In some embodiments, the gRNA that recognizes the re-editable template inserted in exon 3 of the CIITA locus comprises 90% identity to TGACTGATGTAAGACTAGTA (SEQ ID NO: 20). In some embodiments, the gRNA that recognizes the re-editable template inserted in exon 3 of the CIITA locus comprises 95% identity to TGACTGATGTAAGACTAGTA (SEQ ID NO: 20). In some embodiments, the gRNA that recognizes the re-editable template inserted in exon 3 of the CIITA locus comprises 100% identity to TGACTGATGTAAGACTAGTA (SEQ ID NO: 20).

### Cells and Applications

In some embodiments, provided herein is an engineered cell comprising a re-editable target locus, wherein the re-editable target locus comprises an exogenous re-editable template sequence with no substantial sequence similarity to any region in the rest of the genome, and wherein the re-editable template sequence is recognizable by a genome editing system.

In some embodiments, the cell is a mammalian cell. In some embodiments, the cell is a human cell. In some embodiments, the cell is a cultured cell. In some embodiments, the cell is a primary cell. In some embodiments, the cell is a non-dividing cell. In some embodiments, the cell is an immune cell. In some embodiments, the cell is a B-cell, T-cell, monocyte, macrophage or NK-cell. In some embodiments, the cell is a B-cell. In some embodiments, the cell is a T-cell. In some embodiments, the cell is a monocyte. In some embodiments, the cell is a macrophage. In some embodiments, the cell is a NK-cell. In some embodiments, the cell is a stem cell or progenitor cell. In some embodiments, the cell is a stem cell. In some embodiments, the cell is a progenitor cell. In some embodiments, the cell is an induced pluripotent stem cell (iPSC).

In some embodiments, provided herein is a cell, wherein the genome editing system is a CRISPR-Cas-related nuclease system, a Transcription Activator-Like Effector Nuclease (TALEN) system, or a Zinc-Finger Nuclease (ZFN) system. In some embodiments, the Cas protein is Cas9, Cas12a or Cas12b. In some embodiments, the Cas protein is an enzymatically dead Cas protein or a nickase. In some embodiments, the Cas protein is Cas9 D10A.

The cell provided herein comprises the re-editable template as described in other sections of the application, flanked by homology arms and introduced to a re-editable locus. In some embodiments, the re-editable template is less than about 500 nt. In some embodiments, the homology arms are between about 50 to 500 nt.

In some embodiments, provided herein is a method of modifying a gene locus in an engineered cell, comprising contacting the engineered cell with a genome editing system, comprising: a nucleic acid encoding a recombinant nuclease protein or a Cas protein, and a guide RNA that specifically recognizes a PAM sequence comprised in the re-editable template, wherein the recombinant nuclease protein or Cas protein is capable of binding the guide RNA and editing the locus.

In some embodiments, the nucleic acid encoding a Cas protein is fused to an adenine or cytosine deaminase, wherein the Cas protein fusion is capable of binding to the guide RNA and base editing the re-editable template.

Generating a Re-Editable Hypoimmune iPSC Cell Line

Hypoimmune induced pluripotent stem cells are cells that lack one or more immune antigens e.g. HLA Class I and II antigens, that can elicit an immune response and are modified to evade phagocytosis or cytotoxicity, for example, by release of granzyme and/or perforin by immune cells, resulting in improved in vivo persistence. Further, hypoimmune cells avoid the need for HLA matching in adoptive cell therapy and thus provide a universal, off-the-shelf source for generation of a variety of therapeutic cell types, reducing costs and labor. Such a cell platform is useful in generating specific cell products for generating specific tissues and organs for regenerative therapy.

### Human Leukocyte Antigen (HLA) Class I and Class II Genes.

In some embodiments, iPSCs are knocked out for one or more genes selected from HLA Class I and HLA Class II genes. In some embodiments, iPSCs are knocked out for one or more genes selected from a group consisting of B2M, A2AR, LAG3, TIM3, TAP1, TAP2, Tapasin, NLRC5, PD1, RFKANK, CIITA, RFX5, and RFXAP.

β-2 microglobulin (B2M): is expressed ubiquitously in somatic cells and is often considered a ‘house-keeping’ gene. It is transcriptionally active in all cells, at varying levels of expression. In some embodiments, hypoimmunogenic iPSC cells of the present invention are deleted or knocked out for the B2M gene, thereby lacking B2M protein expression. In some embodiments, hypoimmunogenic iPSC cells of the present invention are defective for the B2M gene, thereby reducing B2M protein expression. In some embodiments, the target locus is B2M.

Class II, major histocompatibility complex, transactivator (CTIIA): CTIIA is a human gene which functions as a HLA Class II gene by activating a transcription factor RFX5. The CIITA protein contains an acidic transcriptional activation domain, 4 leucine rich repeats (LRRs) and a GTP binding domain. CTIIA binds GTP and translocates to the nucleus where it promotes Class II HLA gene transcription. CTIIA expression is induced by IFN-gamma. In some embodiments, the target locus is CIITA.

The present invention introduces a re-editable template that knocks out expression of B2M or CIITA, while integrating a unique sequence that can be used to further modify the locus, e.g., by using unique gRNA. Introduction of a re-editable template in the B2M or CIITA loci disrupts wild-type gene expression and results in truncation of the coding region.

Genetically engineering hypoimmune cells requires preventing cell surface expression of Class I and Class II HLA genes, e.g. preventing B2M localization to the cell surface is important to reduce HLA Class I molecules from presenting alloantigens. In some embodiments, Class I and Class II genes are knocked out and replaced with a re-editable template, for example, induced pluripotent stem cells (iPSCs) are engineered to knockout exemplary HLA Class I and Class II genes and generate re-editable B2M and CIITA loci that are subsequently modifiable. For example, a ‘housekeeping gene’ locus (e.g. B2M) is subsequently used to express a protein coding gene of interest from that locus in the future in all somatic cells. In some embodiments, a CIITA locus is used to express a gene of interest in cell types that express HLA Class II antigen. In some embodiments, the loci are modified with a re-editable template that does not include a protein-coding exogenous gene.

In some embodiments, the re-editable template comprises a chimeric antigen receptor (CAR) gene for CAR-T therapy. Chimeric antigen receptor T cells are cells that are genetically engineered to express a new receptor so they can bind to cancer cells and kill them. In some embodiments, the present invention provides hypoimmune pluripotent stem cells containing a nucleic acid encoding CAR. In some embodiments, CAR-expressing hypoimmune cells are homozygous null for B2M gene and/or CIITA gene. Each kind of CAR T cell therapy is made to fight a specific kind of cancer antigen, expressed in different cells. One of the applications of the present invention is that it provides a facile method to rapidly produce cells for CAR T therapy targeting for example, different antigens.

Provided in Tables 1-4 below are guide RNA sequences that direct a double stranded break in exemplary loci, guide RNA sequences that recognize a re-editable template for subsequent editing, re-editable loci and corresponding wild-type loci.

TABLE 1
B2M exon 1
Guide RNA that directs double-stranded break in B2M Exon 1

| Name | Guide RNA sequence | PAM |
|---|---|---|
| B2Mex1-sgRNA-1 | GAGTAGCGCGAGCACAGCTA | AGG |
| | (SEQ ID NO: 1) | |
| B2Mex1-sgRNA-2 | AGGGTAGGAGAGACTCACGC | TGG |
| | (SEQ ID NO: 2) | |
| B2Mex1-sgRNA-3 | GGCCGAGATGTCTCGCTCCG | TGG |
| | (SEQ ID NO: 3) | |

Guide RNA that recognizes Re-editable Template

| | | |
|---|---|---|
| B2Mex1-RE-sgRNA1 | GAGCGTGTGACTAGCTGTAC | TGG |
| | (SEQ ID NO: 4) | |

| | |
|---|---|
| Re-editable B2M | ATTCCTGAAGCTGACAGCATTCGGGCCGAGATGTCCAGGTCCTAATGATTAGC |
| Locus after integration | TGTGCTCGCCCTGCTCTCTCTGTCTGGCCTGGAGGCTATTCAGCGTGAGTCTC |
| (160 nt (35 nt-58 nt- | TCCTACCCTCCCGCTCTGGTCCTTCCTCTCCCGCTCTGCACCCTCTGTGGCCC |
| 67 nt)) | T (SEQ ID NO: 5). |
| | Sequence in bold indicates Re-editable template. |
| B2M KO Re-editable | AAAACGGGAAAGTCCCTCTCTCTAACCTGGCACTGCGTCGCTGGCTTGGAGAC |
| Exon 1 Template | AGGTGACGGTCCCTGCGGGCCTTGTCCTGATTGGCTGGGCACGCGTTTAATAT |
| (codon-optimized) | AAGTGGAGGCGTCGCGCTGGCGGGCATTCCTGAAGCTGACAGCATTCGGGCCG |
| | AGATGTCGTAGAGCGTGTGACTAGCTGTACTGGAGCTGTGAAGCTAATCCGGT |
| | CTGGAAGCCATTCAGCGTGAGTCTCTCCTACCCTCCCGCTCTGGTCCTTCCTC |
| | TCCCGCTCTGCACCCTCTGTGGCCCTCGCTGTGCTCTCTCGCTCCGTGACTTC |
| | CCTTCTCCAAGTTCTCCTTGGT |
| | (SEQ ID NO: 6) |
| | Sequence in bold indicates Re-editable template. Flanking |
| | sequences indicate homology arms. |
| WT B2M Exon 1 | ATTCCTGAAGCTGACAGCATTCGGGCCGAGATGTCTCGCTCCGTGGCCTTAGC |
| Locus | TGTGCTCGCGCTACTCTCTCTTTCTGGCCTGGAGGCTATCCAGCGTGAGTCTC |
| | TCCTACCCTCCCGCTCTGGTCCTTCCTCTCCCGCTCTGCACCCTCTGTGGCCC |
| | T (SEQ ID NO: 22) |
| | Sequence in bold indicates WT template that is knocked out. |

TABLE 2
B2M exon 2
Guide RNA that directs double-stranded break in B2M Exon 2

| Name | Guide RNA sequence | PAM |
|---|---|---|
| B2Mex2-sgRNA-1 | AAGTCAACTTCAATGTCGGA (SEQ ID | TGG |
| | NO: 7) | |
| B2Mex2-sgRNA-2 | AGTCACATGGTTCACACGGC (SEQ ID | AGG |
| | NO: 8) | |
| B2Mex2-sgRNA-3 | ACTTGTCTTTCAGCAAGGAC (SEQ ID | TGG |
| | NO: 9) | |

Guide RNA that recognizes Re-editable Template

| | | |
|---|---|---|
| B2Mex2-RE-sgRNA1 | CCTAGATCCAATAGTAGAGT (SEQ ID | AGG |
| | NO: 10) | |
| B2Mex2-RE-sgRNA2 | GGTCACGTGGTTCACCCTAC (SEQ ID | AGG |
| | NO: 11) | |

| | |
|---|---|
| Re-editable B2M | AAATGTAAACACTTGGTGCCTGATATAGCTTGACACCAAGTTAGCCCCAAGTGA |
| Locus after | AATACCCTGGCAATATTAATGTGTCTTTTCCCGATATTCCTCAGGTACCCCCTA |
| integration | GATCCAATAGTAGAGTAGGTGACCAGCCTAGAACGGAGCCTGTAGGGTGAACCA |
| (160 nt (35 nt-58 nt- | CGTGACCCTGTAACAGTGGGGTAAGTCTTACATTCTTTTGTAAGCTGCTGAAAG |
| 67 nt)) | TTGTGTATGAGTAGTCATATCATAAAGCTGCTTTGATATAAAAAAGGTCTATGG |
| | CCATACTACCC (SEQ ID NO: 12). |
| | Sequence in bold indicates Re-editable template. |
| WT B2M Exon 2 | AAATGTAAACACTTGGTGCCTGATATAGCTTGACACCAAGTTAGCCCCAAGTGA |
| | AATACCCTGGCAATATTAATGTGTCTTTTCCCGATATTCCTCAGGTACTCCAAA |
| | GATTCAGGTTTACTCACGTCATCCAGCAGAGAATGGAAAGTCAAATTTCCTGAA |
| | TTGCTATGTGTCTGGGTTTCATCCATCCGACATTGAAGTTGACTTACTGAAGAA |
| | TGGAGAGAGAATTGAAAAAGTGGAGCATTCAGACTTGTCTTTCAGCAAGGACTG |
| | GTCTTTCTATCTCTTGTACTACACTGAATTCACCCCCACTGAAAAAGATGAGTA |
| | TGCCTGCCGTGTGAACCATGTGACTTTGTCACAGCCCAAGATAGTTAAGTGGGG |
| Locus | TAAGTCTTACATTCTTTTGTAAGCTGCTGAAAGTTGTGTATGAGTAGTCATATC |
| | ATAAAGCTGCTTTGATATAAAAAAGGTCTATGGCCATACTACCC (SEQ ID |
| | NO: 23) |
| | Sequence in bold indicates WT template that is knocked out. |

TABLE 3
CIITA exon 2
Guide RNA that directs double-stranded break in CIITA Exon 2

| Name | Guide RNA sequence | PAM |
|---|---|---|
| CIITAex2-sgRNA-1 | ATGGAGTTGGGGCCCCTAGA (SEQ ID NO: 13) | AGG |
| CIITAex2-sgRNA-2 | CTACCACTTCTATGACCAGA (SEQ ID NO: 14) | TGG |
| CIITAex2-sgRNA-3 | GTGGCACACTGTGAGCTGCC (SEQ ID NO: 15) | TGG |

Guide RNA that recognizes Re-editable Template

| | | |
|---|---|---|
| CIITAex2-RE- | GTGACCCCTATAATGAGACC (SEQ ID NO: 16) | TGG |
| sgRNA-1 | | |
| CIITAex2-RE- | CAGTTCCCAGTGTGCTACCA (SEQ ID NO: 17) | TGG |
| sgRNA-2 | | |

| | |
|---|---|
| Re-editable CIITA | TTCTGCCTCTTTCCAACACCCTGTGAGGTGACTGAGCATTGTCTTCCCTCCCAGG |
| Locus after | CAGTTCCCAGTGTGCTACCATGGAGTTGTGACCCCTATAATGAGACCTGGCTGGA |
| integration | GAAGAAGAGATTGAGCTCTACTCAGGTGGGCCCTCCTCC (SEQ ID NO: 18). |
| | Sequence in bold indicates Re-editable template. |
| WT CIITA Exon 2 | TTCTGCCTCTTTCCAACACCCTGTGAGGTGACTGAGCATTGTCTTCCCTCCCAGG |
| Locus | CAGCTCACAGTGTGCCACCATGGAGTTGGGGCCCCTAGAAGGTGGCTACCTGGAG |
| | CTTCTTAACAGCGATGCTGACCCCCTGTGCCTCTACCACTTCTATGACCAGATGG |
| | ACCTGGCTGGAGAAGAAGAGATTGAGCTCTACTCAGGTGGGCCCTCCTCC (SEQ |
| | ID NO: 24) |
| | Sequence in bold indicates WT template that is knocked out. |

TABLE 4
CIITA exon 3
Guide RNA that directs double-stranded break in CIITA Exon 3

| | | |
|---|---|---|
| CIITAex3- | AGGCTGTTGTGTGACATGGA (SEQ ID NO: 19) | AGG |
| sgRNA-O-CT | | |

Guide RNA that recognizes Re-editable Template

| | | |
|---|---|---|
| CIITAex3-RE | TGACTGATGTAAGACTAGTA (SEQ ID NO: 20) | AGG |
| sgRNA-1 | | |

| | |
|---|---|
| Re-editable | AAATTTCCTTCTTCATCCAAGGGACTTTTCCTCCCAGAACCCGACACAGACACCATCAA |
| CIITA Locus | CTGCGACCAGTTCAGCTGACTGATGTAAGACTAGTAAGGTGATGAAGAGACCAGGGAGG |
| after integration | CTTATGCCAATATCGGTGAGGAAGCACCTGAGCCCAGAAAAGGACAATCAAGGGCAAGA |
| | GTTCTTTGCTGCCACTTGTCA (SEQ ID NO: 21). |
| | Sequence in bold indicates Re-editable template. |
| WT CIITA Exon | AAATTTCCTTCTTCATCCAAGGGACTTTTCCTCCCAGAACCCGACACAGACACCATCAA |
| 3 Locus | CTGCGACCAGTTCAGCAGGCTGTTGTGTGACATGGAAGGTGATGAAGAGACCAGGGAGG |
| | CTTATGCCAATATCGGTGAGGAAGCACCTGAGCCCAGAAAAGGACAATCAAGGGCAAGA |
| | GTTCTTTGCTGCCACTTGTCA (SEQ ID NO: 25) |
| | Sequence in bold indicates WT template that is knocked out. |

### EXAMPLES

### Example 1. Exemplary Re-Editable Template Design and Integration into a Target Locus

This example illustrates exemplary re-editable template integration into a target locus to form a re-editable target locus.

Briefly, in this example, gRNAs are designed to direct a double stranded break to insert a re-editable template in exemplary target gene loci: B2M exon 1, B2M exon 2, CIITA exon 2 and CIITA exon 3.

A unique re-editable template is designed that has no substantial sequence similarity to any region in the genome, and wherein the template sequence is recognizable by a genome editing system, e.g. CRISPR-Cas, Transcription Activator-Like Effector Nucleases (TALEN), or Zinc-finger Nuclease (ZFN) system. In some embodiments, a re-editable template is a sequence that is less than about 100 nt. In some embodiments, wherein the genome editing tool is CRISPR-Cas, the re-editable template comprises a Protospacer Adjacent Motif (PAM) recognizable by a specific Cas nuclease. In some embodiments, the re-editable template comprises a 5′-NGG-3′ PAM and the nuclease is SpCas9.

In this example, a gRNA spacer was designed to be about 20 nt followed by 5′-NGG-3′ PAM sequence that has greater than 2 mismatches for SpCas9. A gRNA checker tool was used with the criteria that the proposed target sequence (20 nt) should have a score of greater than 50% for predicted on-target efficiency and no protein-coding genes with less than 3 mismatches in the predicted off-target list. No target site with 100% homology (e.g. 0 mismatches) was identified in the human reference genome.

In some embodiments, off-target editing is absent.

Using gene editing tools, for example CRISPR-Cas, the re-editable template flanked by site-specific homology arms is integrated into the target gene locus by homology directed recombination. In some embodiments, the homology arms are between about 50 to 500 nt. In some embodiments, the homology arms are about 100 nt. The homology arms are designed for each target gene locus taking into consideration GC content, structural repeats, etc. Provided below are exemplary guide RNAs and re-editable target loci.

### Re-Editable B2M Exon 1 Locus

In this example, in some embodiments, the gRNA that directs a double-stranded break to insert a re-editable template in a B2M exon 1 locus comprises 70%, 80%, 90%, 95%, 99% or 100% identity to GAGTAGCGCGAGCACAGCTA (SEQ ID NO: 1), AGGGTAGGAGAGACTCACGC (SEQ ID NO: 2) or GGCCGAGATGTCTCGCTCCG (SEQ ID NO: 3). In some embodiments, the gRNA that recognizes the re-editable template inserted in a B2M exon 1 locus comprises 70%, 80%, 90%, 95%, 99% or 100% identity to GAGCGTGTGACTAGCTGTAC (SEQ ID NO: 4).

The re-editable locus is defined by a sequence comprising 70%, 80%, 90%, 95%, 99% or 100% identity to

(SEQ ID NO: 5)
ATTCCTGAAGCTGACAGCATTCGGGCCGAGATGTCCAGGTCCTAATGAT
TAGCTGTGCTCGCCCTGCTCTCTCTGTCTGGCCTGGAGGCTATTCAGCG
TGAGTCTCTCCTACCCTCCCGCTCTGGTCCTTCCTCTCCCGCTCTGCAC
CCTCTGTGGCCCT,
or
(SEQ ID NO: 6)
AAAACGGGAAAGTCCCTCTCTCTAACCTGGCACTGCGTCGCTGGCTTGG
AGACAGGTGACGGTCCCTGCGGGCCTTGTCCTGATTGGCTGGGCACGCG
TTTAATATAAGTGGAGGCGTCGCGCTGGCGGGCATTCCTGAAGCTGACA
GCATTCGGGCCGAGATGTCGTAGAGCGTGTGACTAGCTGTACTGGAGCT
GTGAAGCTAATCCGGTCTGGAAGCCATTCAGCGTGAGTCTCTCCTACCC
TCCCGCTCTGGTCCTTCCTCTCCCGCTCTGCACCCTCTGTGGCCCTCGC
TGTGCTCTCTCGCTCCGTGACTTCCCTTCTCCAAGTTCTCCTTGGT.

The sequence in bold represents the re-editable template, while the flanking sequences are the homology arms.

### Re-Editable B2M Exon 2 Locus

In some embodiments, the gRNA that directs a double-stranded break to insert a re-editable template in a B2M exon 2 locus comprises 70%, 80%, 90%, 95%, 99% or 100% identity to AAGTCAACTTCAATGTCGGA (SEQ ID NO: 7), AGTCACATGGTTCACACGGC (SEQ ID NO: 8), or ACTTGTCTTTCAGCAAGGAC (SEQ ID NO: 9).

The gRNA that recognizes the re-editable template inserted in a B2M exon 2 locus comprises 70%, 80%, 90%, 95%, 99% or 100% identity to CCTAGATCCAATAGTAGAGT (SEQ ID NO: 10) or GGTCACGTGGTTCACCCTAC (SEQ ID NO: 11).

The re-editable locus is defined by a sequence comprising 70%, 80%, 90%, 95%, 99% or 100% identity to AAATGTAAACACTTGGTGCCTGATATAGCTTGACACCAAGTTAGCCCCAAGTGA AATACCCTGGCAATATTAATGTGTCTTTTCCCGATATTCCTCAGGTACCCCCTAG ATCCAATAGTAGAGTAGGTGACCAGCCTAGAACGGAGCCTGTAGGGTGAAC CACGTGACCCTGTAACAGTGGGGTAAGTCTTACATTCTTTTGTAAGCTGCTGAA AGTTGTGTATGAGTAGTCATATCATAAAGCTGCTTTGATATAAAAAAGGTCTATG GCCATACTACCC (SEQ ID NO: 12). The sequence in bold represents the re-editable template, while the flanking sequences are the homology arms.

### Re-Editable CIITA Exon 2 Locus

In the embodiments, the gRNA that directs a double-stranded break to insert a re-editable template in exon 2 of the CIITA locus comprises 70%, 80%, 90%, 95%, 99% or 100% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15).

In some embodiments, the gRNA that recognizes the re-editable template inserted in a CTIIA exon 2 locus comprises 70%, 80%, 90%, 95%, 99% or 100% identity to GTGACCCCTATAATGAGACC (SEQ ID NO: 16) or CAGTTCCCAGTGTGCTACCA (SEQ ID NO: 17).

The re-editable locus is defined by a sequence comprising 70%, 80%, 90%, 95%, 99% or 100% identity to

(SEQ ID NO: 18)
TTCTGCCTCTTTCCAACACCCTGTGAGGTGACTGAGCATTGTCTTCCCT
CCCAGGCAGTTCCCAGTGTGCTACCATGGAGTTGTGACCCCTATAATGA
GACCTGGCTGGAGAAGAAGAGATTGAGCTCTACTCAGGTGGGCCCTCCT
CC.

The sequence in bold represents the re-editable template, while the flanking sequences are the homology arms. Introduction of a re-editable template in the CIITA exon 2 locus removes an alternative splice site and disrupts wild-type gene expression.

### Re-Editable CIITA Exon 3 Locus

The gRNA that directs a double-stranded break to insert a re-editable template in exon 3 of the CIITA locus comprises 70%, 80%, 90%, 95%, 99% or 100% identity to AGGCTGTTGTGTGACATGGA (SEQ ID NO: 19).

The gRNA that recognizes the re-editable template inserted in exon 3 of the CIITA locus comprises 70%, 80%, 90%, 95%, 99% or 100% identity to TGACTGATGTAAGACTAGTA (SEQ ID NO: 20).

The re-editable locus is defined by a sequence comprising 70%, 80%, 90%, 95%, 99% or 100% identity to

(SEQ ID NO: 21)
AAATTTCCTTCTTCATCCAAGGGACTTTTCCTCCCAGAACCCGACACAG
ACACCATCAACTGCGACCAGTTCAGCTGACTGATGTAAGACTAGTAAGG
TGATGAAGAGACCAGGGAGGCTTATGCCAATATCGGTGAGGAAGCACCT
GAGCCCAGAAAAGGACAATCAAGGGCAAGAGTTCTTTGCTGCCACTTGT
CA.

The sequence in bold represents the re-editable template, while the flanking sequences are the homology arms.

In summary, the unique re-editable templates designed in this example, are integrated into specific target gene loci using gRNAs designed for each target, to form re-editable target loci, which are recognizable by genome editing tools. The re-editable loci are editable by a different guide RNA from the one used to insert the template. Provided herein are guide RNAs that specifically recognize re-editable loci for subsequent genome modification. Based on the methods provided herein, one of ordinary skill can design a unique re-editable template that has no substantial sequence similarity with any other region of the genome and a gRNA specific for any locus to generate any re-editable target locus that is specifically recognizable by one or more genome editing systems, for subsequent modification.

### Example 2: Generation of Exemplary Re-Editable Cells

This example illustrates generation of exemplary re-editable cells, comprising an exogenous re-editable template flanked by two homology arms integrated into a target locus by homologous recombination as described in Example 1.

Briefly, the re-editable templates designed in Example 1 are introduced into cells by methods known in the art, e.g., transfection, transduction, transformation, conjugation, and includes technologies such as electroporation of nucleic acids e.g., vectors, plasmids, minicircles, circular single-stranded DNA, linear double-stranded or linear single-stranded DNA, biolistic methods, microinjection, among others.

The homology arms designed in Example 1 for each specific locus flank the re-editable template and introduce the exogenous re-editable template sequence into the target locus by homologous recombination, thereby generating a cell comprising a re-editable template locus, which is recognizable by one or more genome editing systems for subsequent modification.

The exemplary cell, of this example, is a human iPSC cell, comprising re-editable Class I and/or Class II HLA gene loci. For example, an iPSC cell line comprising a B2M re-editable loci is generated by the methods of this example. In another embodiment, an iPSC cell comprising CIITA re-editable loci is generated by the methods of this example. In another embodiment, an iPSC cell comprising both B2M re-editable and CIITA re-editable loci is generated by the methods of this example.

Based on the methods described in this example, one of ordinary skill can generate any cell comprising a unique re-editable template that has no substantial sequence similarity with any other region of the genome to generate any re-editable target locus that is specifically recognizable by one or more genome editing systems, for subsequent modification. For example, the cell is a mammalian cell, human cell, cultured cell, primary cell, non-dividing cell or immune cell. The exemplary immune cell is B-cell, T-cell, monocyte, macrophage or NK-cell. In some embodiments, the cell is a stem cell. In some embodiments, the cell is a cell line used to generate a platform that is rapidly modifiable for various uses.

### Example 3: Generating a Hypoimmune iPSC Cell Line

Briefly, in this example, the methods described in Example 1 and 2 are used to generate a hypoimmune iPSC cell line.

Induced pluripotent stem cells (iPSCs) are engineered to knockout exemplary HLA Class I and Class II genes and generate re-editable B2M and CIITA loci that are subsequently modifiable.

In exemplary embodiments, the subsequent modification uses a gRNA guided Cas nuclease to generate a double-stranded break and integrate a protein-coding gene. In some embodiments, the protein coding gene is a cloaking gene that protects cells from recognition by NK cells, generating hypoimmune iPSCs. In some embodiments, the protein-coding gene triggers cell termination in the presence of an inducing molecule. In some embodiments, the protein coding gene expresses one or more proteins that increase efficacy of the CAR product.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will recognize, or be able to ascertain using no more than routine experimentation, many equivalents to the specific inventive embodiments described herein. It is, therefore, to be understood that the foregoing embodiments are presented by way of examples only and that, within the scope of the appended claims and equivalents thereto, inventive embodiments may be practiced otherwise than as specifically described and claimed.

1. A method of engineering a re-editable target locus in a cell, comprising inserting an exogenous re-editable template sequence to a target locus, wherein the template sequence has no substantial sequence similarity to any region in the genome, and wherein the template sequence is recognizable by a genome editing system.

2. A method of engineering a re-editable cell, comprising
introducing an exogenous re-editable template sequence flanked by two homology arms such that the re-editable template sequence is inserted into the target locus by homologous recombination, wherein the template sequence has no substantial sequence similarity to any region in the genome, and wherein the template sequence is recognizable by a genome editing system.

3. The method of any one of the preceding claims, wherein the genome editing system is a CRISPR-Cas-related nuclease system, a Transcription Activator-Like Effector Nuclease (TALEN) system, or a Zinc-finger Nuclease (ZFN) system.

4. The method of claim 3, wherein the Cas protein is Cas9, Cas12a or Cas12b.

5. The method of claim 4, wherein the Cas protein is an enzymatically dead Cas protein or a nickase.

6. The method of claim 5, wherein the Cas protein is Cas9 D10A or Cas9 H480A.

7. The method of claim 3, wherein the Cas protein, TALEN or ZFN is fused to a FokI nuclease or related nuclease domain.

8. The method of any one of the preceding claims, wherein each homology arm flanking the re-editable template is between about 50 to 500 nt.

9. The method of claim 8, wherein the homology arm is about 100 nt.

10. The method of any one of the preceding claims, wherein the re-editable template is less than about 500 nt.

11. The method of any one of the preceding claims, wherein the re-editable template is between about 10-500 nt.

12. The method of claim 11, wherein the re-editable template is about 100 nt.

13. The method of claim 11, wherein the re-editable template is about 500 nt.

14. The method of any one of the preceding claims, wherein the re-editable template comprises a Protospacer Adjacent Motif (PAM).

15. The method of claim 10, wherein the re-editable template comprises a 5′-NGG-3′ PAM and the nuclease is Cas9.

16. The method of any one of the preceding claims, wherein any coding region in the genome contains at least 3 mismatches relative to the sequence in the re-editable template recognizable by a guide RNA.

17. The method of claim 16, wherein off-target editing is absent.

18. The method of any one of the preceding claims, wherein the cell is a mammalian cell.

19. The method of claim 18, wherein the cell is a human cell.

20. The method of claim 18, wherein the cell is a cultured cell.

21. The method of claim 18, wherein the cell is a primary cell.

22. The method of claim 18, wherein the cell is a non-dividing cell.

23. The method of claim 18, wherein the cell is an immune cell.

24. The method of claim 23, wherein the cell is a B-cell, T-cell, monocyte, macrophage or NK-cell.

25. The method of claim 18, wherein the cell is a stem cell or progenitor cell.

26. The method of claim 25, wherein the cell is an induced pluripotent stem cell (iPSC).

27. The method of any one of the preceding claims, wherein the target locus is at least one locus selected from the group consisting of a ubiquitously expressed gene, cell division related gene, and gene with expression restricted to specific cell types.

28. The method of claim 27, wherein the target locus is a Class I or Class II HLA gene.

29. The method of claim 28, wherein the target locus is B2M.

30. The method of claim 29, wherein the gRNA that directs a double-stranded break to insert a re-editable template in a B2M exon 1 locus comprises 70%, 80%, 90%, 95%, 99% or 100% identity to GAGTAGCGCGAGCACAGCTA (SEQ ID NO: 1), AGGGTAGGAGAGACTCACGC (SEQ ID NO: 2) or GGCCGAGATGTCTCGCTCCG (SEQ ID NO: 3).

31. The method of claim 30, wherein the re-editable locus is defined by a sequence comprising 70%, 80%, 90%, 95%, 99% or 100% identity to
(SEQ ID NO: 5)
ATTCCTGAAGCTGACAGCATTCGGGCCGAGATGTCCAGGTCCTAATGAT
TAGCTGTGCTCGCCCTGCTCTCTCTGTCTGGCCTGGAGGCTATTCAGCG
TGAGTCTCTCCTACCCTCCCGCTCTGGTCCTTCCTCTCCCGCTCTGCAC
CCTCTGTGGCCCT,
or
(SEQ ID NO: 6)
AAAACGGGAAAGTCCCTCTCTCTAACCTGGCACTGCGTCGCTGGCTTGG
AGACAGGTGACGGTCCCTGCGGGCCTTGTCCTGATTGGCTGGGCACGCG
TTTAATATAAGTGGAGGCGTCGCGCTGGCGGGCATTCCTGAAGCTGACA
GCATTCGGGCCGAGATGTCGTAGAGCGTGTGACTAGCTGTACTGGAGCT
GTGAAGCTAATCCGGTCTGGAAGCCATTCAGCGTGAGTCTCTCCTACCC
TCCCGCTCTGGTCCTTCCTCTCCCGCTCTGCACCCTCTGTGGCCCTCGC
TGTGCTCTCTCGCTCCGTGACTTCCCTTCTCCAAGTTCTCCTTGGT.

32. The method of claim 29, wherein the gRNA that directs a double-stranded break to insert a re-editable template in a B2M exon 2 locus comprises 70%, 80%, 90%, 95%, 99% or 100% identity to AAGTCAACTTCAATGTCGGA (SEQ ID NO: 7), AGTCACATGGTTCACACGGC (SEQ ID NO: 8), or ACTTGTCTTTCAGCAAGGAC (SEQ ID NO: 9).

33. The method of claim 29, wherein the gRNA that recognizes the re-editable template inserted in a B2M exon 2 locus comprises 70%, 80%, 90%, 95%, 99% or 100% identity to CCTAGATCCAATAGTAGAGT (SEQ ID NO: 10) or GGTCACGTGGTTCACCCTAC (SEQ ID NO: 11), or that recognizes the re-editable template inserted in a B2M exon 1 locus comprises 70%, 80%, 90%, 95%, 99% or 100% identity to GAGCGTGTGACTAGCTGTAC (SEQ ID NO: 4).

34. The method of claim 33, wherein the re-editable locus is defined by a sequence comprising 70%, 80%, 90%, 95%, 99% or 100% identity to
(SEQ ID NO: 12)
AAATGTAAACACTTGGTGCCTGATATAGCTTGACACCAAGTTAGCCCCA
AGTGAAATACCCTGGCAATATTAATGTGTCTTTTCCCGATATTCCTCAG
GTACCCCCTAGATCCAATAGTAGAGTAGGTGACCAGCCTAGAACGGAGC
CTGTAGGGTGAACCACGTGACCCTGTAACAGTGGGGTAAGTCTTACATT
CTTTTGTAAGCTGCTGAAAGTTGTGTATGAGTAGTCATATCATAAAGCT
GCTTTGATATAAAAAAGGTCTATGGCCATACTACCC.

35. The method of claim 28, wherein the target locus is CIITA.

36. The method of claim 35, wherein the gRNA that directs a double-stranded break to insert a re-editable template in exon 2 of the CIITA locus comprises 70%, 80%, 90%, 95%, 99% or 100% identity to ATGGAGTTGGGGCCCCTAGA (SEQ ID NO: 13), CTACCACTTCTATGACCAGA (SEQ ID NO: 14) or GTGGCACACTGTGAGCTGCC (SEQ ID NO: 15).

37. The method of claim 35, wherein the gRNA that recognizes the re-editable template inserted in exon 2 of the CIITA locus comprises 70%, 80%, 90%, 95%, 99% or 100% identity to GTGACCCCTATAATGAGACC (SEQ ID NO: 16) or CAGTTCCCAGTGTGCTACCA (SEQ ID NO: 17).

38. The method of claim 36, wherein the re-editable locus is defined by a sequence comprising 70%, 80%, 90%, 95%, 99% or 100% identity to
(SEQ ID NO: 18)
TTCTGCCTCTTTCCAACACCCTGTGAGGTGACTGAGCATTGTCTTCCCT
CCCAGGCAGTTCCCAGTGTGCTACCATGGAGTTGTGACCCCTATAATGA
GACCTGGCTGGAGAAGAAGAGATTGAGCTCTACTCAGGTGGGCCCTCCT
CC.

39. The method of claim 38, wherein the gRNA that directs a double-stranded break to insert a re-editable template in exon 3 of the CIITA locus comprises 70%, 80%, 90%, 95%, 99% or 100% identity to AGGCTGTTGTGTGACATGGA (SEQ ID NO: 19).

40. The method of claim 35, wherein the gRNA that recognizes the re-editable template inserted in exon 3 of the CIITA locus comprises 70%, 80%, 90%, 95%, 99% or 100% identity to TGACTGATGTAAGACTAGTA (SEQ ID NO: 20).

41. The method of claim 40, wherein the re-editable locus is defined by a sequence comprising 70%, 80%, 90%, 95%, 99% or 100% identity to
(SEQ ID NO: 21)
AAATTTCCTTCTTCATCCAAGGGACTTTTCCTCCCAGAACCCGACACAG
ACACCATCAACTGCGACCAGTTCAGCTGACTGATGTAAGACTAGTAAGG
TGATGAAGAGACCAGGGAGGCTTATGCCAATATCGGTGAGGAAGCACCT
GAGCCCAGAAAAGGACAATCAAGGGCAAGAGTTCTTTGCTGCCACTTGT
CA.

42. The method of any one of the preceding claims, wherein the re-editable template comprises an exogenous protein-coding gene.

43. The method of claim 42, wherein the exogenous gene is an immune modulatory or cloaking gene.

44. The method of claim 42, wherein the exogenous gene expresses a protein that leads to cell death, wherein the gene is selected from the group consisting of HSV-TK, iCaspase8 and iCaspase9.

45. The method of any one of the preceding claims, wherein the re-editable template comprises a chimeric antigen receptor (CAR) gene.

46. An isolated cell engineered by the method of any one of the preceding claims.

47. An engineered cell comprising a re-editable target locus, wherein the re-editable target locus comprises an exogenous re-editable template sequence with no substantial sequence similarity to any region in the rest of the genome, and wherein the re-editable template sequence is recognizable by a genome editing system.

48. The cell of claim 47, wherein the genome editing system is a CRISPR-Cas-related nuclease system, a Transcription Activator-Like Effector Nuclease (TALEN) system, or a Zinc-Finger Nuclease (ZFN) system.

49. The cell of claim 48, wherein a Cas protein of the CRISPR-Cas-related nuclease system is Cas9, Cas12a or Cas12b.

50. The cell of claims 47-49, wherein the Cas protein is an enzymatically dead Cas protein or a nickase.

51. The cell of claim 50, wherein the Cas protein is Cas9 D10A or Cas9 H840A.

52. The cell of any one of the preceding claims, wherein the re-editable template is flanked by homology arms, wherein the homology arms are between about 50 to 500 nt.

53. The cell of claim 52, wherein the homology arms are about 100 nt.

54. The cell of any one of the preceding claims, wherein the re-editable template is less than about 500 nt.

55. The cell of any one of the preceding claims, wherein the re-editable template is less than about 100 nt.

56. The cell of any one of the preceding claims, wherein the re-editable template comprises a Protospacer Adjacent Motif (PAM).

57. The cell of claim 56, wherein the re-editable template comprises a 5′-NGG-3′ PAM and the nuclease is Cas9.

58. The cell of any one of the preceding claims, wherein a coding region in the genome recognizable by a guide RNA contains at least 3 mismatches.

59. The cell of claim 58, wherein off-target editing is absent.

60. The cell of any one of the preceding claims, wherein the cell is a mammalian cell.

61. The cell of claim 60, wherein the cell is a human cell.

62. The cell of claim 60, wherein the cell is a cultured cell.

63. The cell of claim 60, wherein the cell is a primary cell.

64. The cell of claim 60, wherein the cell is a non-dividing cell.

65. The cell of claim 60, wherein the cell is an immune cell.

66. The cell of claim 65, wherein the cell is a B-cell, T-cell, monocyte, macrophage or NK-cell.

67. The cell of claim 60, wherein the cell is a stem cell.

68. The cell of claim 67, wherein the cell is an induced pluripotent stem cell (iPSC).

69. The cell of any one of the preceding claims, wherein the target locus encodes an immune gene.

70. The cell of claim 69, wherein the target locus encodes a Class I or Class II HLA gene.

71. The cell of claim 70, wherein the target locus is B2M.

72. The cell of claim 71, wherein the gRNA that directs a double-stranded break to insert a re-editable template in a B2M exon 1 locus comprises 70%, 80%, 90%, 95%, 99% or 100% identity to GAGTAGCGCGAGCACAGCTA (SEQ ID NO: 1), AGGGTAGGAGAGACTCACGC (SEQ ID NO: 2) or GGCCGAGATGTCTCGCTCCG (SEQ ID NO: 3).

73. The cell of claim 72, wherein the re-editable locus is defined by a sequence comprising 70%, 80%, 90%, 95%, 99% or 100% identity to
(SEQ ID NO: 5)
ATTCCTGAAGCTGACAGCATTCGGGCCGAGATGTCCAGGTCCTAATGAT
TAGCTGTGCTCGCCCTGCTCTCTCTGTCTGGCCTGGAGGCTATTCAGCG
TGAGTCTCTCCTACCCTCCCGCTCTGGTCCTTCCTCTCCCGCTCTGCAC
CCTCTGTGGCCCT,
or
(SEQ ID NO: 6)
AAAACGGGAAAGTCCCTCTCTCTAACCTGGCACTGCGTCGCTGGCTTGG
AGACAGGTGACGGTCCCTGCGGGCCTTGTCCTGATTGGCTGGGCACGCG
TTTAATATAAGTGGAGGCGTCGCGCTGGCGGGCATTCCTGAAGCTGACA
GCATTCGGGCCGAGATGTCGTAGAGCGTGTGACTAGCTGTACTGGAGCT
GTGAAGCTAATCCGGTCTGGAAGCCATTCAGCGTGAGTCTCTCCTACCC
TCCCGCTCTGGTCCTTCCTCTCCCGCTCTGCACCCTCTGTGGCCCTCGC
TGTGCTCTCTCGCTCCGTGACTTCCCTTCTCCAAGTTCTCCTTGGT.

74. The cell of claim 73, wherein the gRNA that recognizes the re-editable template inserted in a B2M exon 1 locus comprises 70%, 80%, 90%, 95%, 99% or 100% identity to GAGCGTGTGACTAGCTGTAC (SEQ ID NO: 4).

75. The cell of claim 73, wherein the gRNA that directs a double-stranded break to insert a re-editable template in a B2M exon 2 locus comprises 70%, 80%, 90%, 95%, 99% or 100% identity to AAGTCAACTTCAATGTCGGA (SEQ ID NO: 7), AGTCACATGGTTCACACGGC (SEQ ID NO: 8), or ACTTGTCTTTCAGCAAGGAC (SEQ ID NO: 9).

76. The cell of claim 73, wherein the gRNA that recognizes the re-editable template inserted in a B2M exon 2 locus comprises 70%, 80%, 90%, 95%, 99% or 100% identity to
(SEQ ID NO: 10)
CCTAGATCCAATAGTAGAGT
or
(SEQ ID NO: 11)
GGTCACGTGGTTCACCCTAC.

77. The cell of claim 76, wherein the re-editable locus is defined by a sequence comprising 70%, 80%, 90%, 95%, 99% or 100% identity to
(SEQ ID NO: 12)
AAATGTAAACACTTGGTGCCTGATATAGCTTGACACCAAGTTAGCCCCA
AGTGAAATACCCTGGCAATATTAATGTGTCTTTTCCCGATATTCCTCAG
GTACCCCCTAGATCCAATAGTAGAGTAGGTGACCAGCCTAGAACGGAGC
CTGTAGGGTGAACCACGTGACCCTGTAACAGTGGGGTAAGTCTTACATT
CTTTTGTAAGCTGCTGAAAGTTGTGTATGAGTAGTCATATCATAAAGCT
GCTTTGATATAAAAAAGGTCTATGGCCATACTACCC.

78. The cell of claim 70, wherein the target locus is CIITA.

79. The cell of claim 78, wherein the gRNA that directs a double-stranded break to insert a re-editable template in exon 2 of the CIITA locus comprises 70%, 80%, 90%, 95%, 99% or 100% identity to ATGGAGTTGGGGCCCCTAGA (SEQ ID NO. 13), CTACCACTTCTATGACCAGA (SEQ ID NO: 14) or GTGGCACACTGTGAGCTGCC (SEQ ID NO: 15).

80. The cell of claim 78, wherein the gRNA that recognizes the re-editable template inserted in exon 2 of CIITA locus comprises 70%, 80%, 90%, 95%, 99% or 100% identity to GTGACCCCTATAATGAGACC (SEQ ID NO: 16) or CAGTTCCCAGTGTGCTACCA (SEQ ID NO: 17).

81. The cell of claim 78, wherein the re-editable locus is defined by a sequence comprising 70%, 80%, 90%, 95%, 99% or 100% identity to
(SEQ ID NO: 18)
TTCTGCCTCTTTCCAACACCCTGTGAGGTGACTGAGCATTGTCTTCCCT
CCCAGGCAGTTCCCAGTGTGCTACCATGGAGTTGTGACCCCTATAATGA
GACCTGGCTGGAGAAGAAGAGATTGAGCTCTACTCAGGTGGGCCCTCCT
CC.

82. The cell of claim 78, wherein the gRNA that directs a double-stranded break to insert a re-editable template in exon 3 of the CIITA locus comprises 70%, 80%, 90%, 95%, 99% or 100% identity to AGGCTGTTGTGTGACATGGA (SEQ ID NO: 19).

83. The cell of claim 78, wherein the gRNA that recognizes the re-editable template inserted in exon 3 of the CIITA locus comprises 70%, 80%, 90%, 95%, 99% or 100% identity to TGACTGATGTAAGACTAGTA (SEQ ID NO: 20).

84. The cell of claim 82, wherein the re-editable locus is defined by a sequence comprising 70%, 80%, 90%, 95%, 99% or 100% identity to
(SEQ ID NO: 21)
AAATTTCCTTCTTCATCCAAGGGACTTTTCCTCCCAGAACCCGACACAG
ACACCATCAACTGCGACCAGTTCAGCTGACTGATGTAAGACTAGTAAGG
TGATGAAGAGACCAGGGAGGCTTATGCCAATATCGGTGAGGAAGCACCT
GAGCCCAGAAAAGGACAATCAAGGGCAAGAGTTCTTTGCTGCCACTTGT
CA.

85. The cell of any one of the preceding claims, wherein the re-editable template comprises an exogenous protein-coding gene.

86. The cell of claim 85, wherein the exogenous protein-coding gene is an immune modulatory or cloaking gene.

87. The cell of claim 85, wherein the exogenous protein-coding gene expresses a protein that leads to cell death.

88. The cell of any one of the preceding claims, wherein the re-editable template comprises a chimeric antigen receptor (CAR) gene.

89. A method of modifying a gene locus in an engineered cell, comprising contacting the engineered cell of claim 46 or 47 with a genome editing system, comprising:
a nucleic acid encoding a recombinant nuclease protein or a Cas protein, and
a guide RNA that specifically recognizes a PAM sequence comprised in the re-editable template,
wherein the recombinant nuclease protein or Cas protein is capable of binding the guide RNA and editing the locus.

90. The method of claim 89, wherein the nucleic acid encoding a Cas protein is fused to an adenine or cytosine deaminase, and wherein the Cas protein fusion is capable of binding to the guide RNA and base editing the re-editable template.

91. A re-editable template sequence, wherein the template sequence has no substantial sequence similarity to any region in the genome, and wherein the template sequence is recognizable by a genome editing system.
